(19) United States
(12) Patent Application Publication
(10) Pub. No.: US 2024/0017001 A1
(43) Pub. Date: Jan. 18, 2024

(54) SYSTEMS, DEVICES, AND METHODS FOR FLUID PUMPING

(71) Applicants: Chaoyoung Lee, Weston, MA (US); Gregory R. Lanier, JR., Merrimack, NH (US); Zhenhua Mao, Andover, MA (US)

(72) Inventors: Chaoyoung Lee, Weston, MA (US); Gregory R. Lanier, JR., Merrimack, NH (US); Zhenhua Mao, Andover, MA (US)

(21) Appl. No.: 18/122,020

(22) Filed: Oct. 2, 2023

Related U.S. Application Data
(60) Provisional application No. 63/320,193, filed on Mar. 15, 2022.

(51) Int. Cl. A61M 5/142

(52) U.S. Cl. CPC A61M 5/14248; A61M 5/14228; A61M 2005/14252

(57) ABSTRACT

A fluid pumping system. The system includes a fluid pump device including a pump housing; an adhesive located on the outside of the pump housing; and an adhesive cover attached to the adhesive; and a vial transfer station, wherein the adhesive cover is attached to the vial transfer station; wherein when the fluid pump device is removed from the vial transfer station, the adhesive cover is removed from the adhesive and the adhesive cover remains attached to the vial transfer station.

### REFERENCE TO CROSS-RELATED APPLICATION(S)

The present application claims priority to U.S. Provisional Patent Application No. 63/320,193 (Attorney Docket No.: ITX-401R) filed on Mar. 15, 2022 and is hereby incorporated herein by reference in its entirety.

### FIELD OF THE DISCLOSURE

The present disclosure relates to fluid delivery, and more particularly, to systems, devices, and methods for fluid pumping.

### BACKGROUND OF THE DISCLOSURE

Many pharmaceutical and non-pharmaceutical medications and/or therapeutics (together referred to herein as “drugs”) are delivered via subcutaneous, intravenous, or inhalation methods. Traditionally, drug and/or medication therapy delivered subcutaneously or intravenously is administered by trained clinicians, typically in a clinical setting. Some exceptions include oral administration including oral inhalation and single injections (e.g., insulin multiple daily injections) which may be patient supervised with guidance from their medical team, i.e., by prescription. However, in these exceptional cases, the medications are by and large administered by the patient based on need, which change hourly and daily. Patient-managed delivery of pharmaceutical and non-pharmaceutical medications and/or therapeutics via subcutaneous, intravenous, or inhalation is therefore limited to very specific circumstances and, to large extent, limited to diabetes therapy, asthma therapies, and other chronic diseases in which the patient is well-trained over a number of years to safely deliver their own therapy. These therapies are also not “one-off” therapies, therefore, the patient/caregiver invests time in learning how to safely administer their therapies, as well as administer them multiple times daily for the rest of their lives. However, many other subcutaneously and/or intravenously and/or inhalation delivered therapies are used for a limited time, with a pre-determined, pre-prescribed dosage and could be delivered by a patient under the prescription and guidance of their physician or healthcare/medical team if the patient and/or their caregiver were given the right tools to ensure safe delivery.

Therapy outcomes improve when patients have access to their therapies in the at-home setting. As well, patient satisfaction improves, which leads to even better clinical outcomes. However, safety is a primary concern and for many of these medications, over delivery may result in serious bodily harm. Further, compliance may be a concern, particularly with therapies that are essential to the patient's health and continued well-being. Thus, for these patients, oftentimes self-administration is not an option given the medical team may lack confidence that the therapy may be safely delivered, per their prescription, to the patient.

As technology advances in both the pharmaceutical and digital space, the potential benefits of turning drug substances into targeted disease management therapy via a precision delivery device that is self-administered by the patient significantly increases. Moving more therapy outside the clinical settings may improve patient health and satisfaction, and at the same time, reduce costs related to clinically-administered therapeutics that may be safely administered by the patient outside the clinical setting. Allowing patients/caregivers to administer their own therapies also increases access to these therapies. Patients would no longer require the availability or access to a clinic or clinical setting. Rather, the patient/caregiver could administer the therapy safely and effectively anywhere, including in the home.

Fixed-volume therapies include inoculations and once-a-day administrations, wherein the patient/caregiver simply delivers the volume or dosage prescribed by a physician, in the manner prescribed. Thus, these therapies differ from, e.g. insulin therapy, since insulin therapy is generally delivered multiple times per day and based on patient needs. Thus, fixed-volume therapies are a good candidate for patient self-administration since there are no decisions made by the patient/caregiver, they simply administer the therapy as directed.

Therefore, safe devices and methods for the precision delivery of drug substances via subcutaneous, intravenous, and/or inhalation delivery methods, that is self-administered by the patient, in an at-home or outside the clinical setting is desired. A system to ensure the safety and compliance of patients in the at-home setting is desired.

Wherefore it is an object of the present disclosure to overcome the above-mentioned shortcomings and drawbacks associated with conventional clinical therapeutic delivery.

### SUMMARY OF THE DISCLOSURE

One aspect of the present disclosure includes a fluid pumping system including a fluid pump device including a pump housing; an adhesive located on the outside of the pump housing; and an adhesive cover attached to the adhesive; and a vial transfer station, wherein the adhesive cover is attached to the vial transfer station; wherein when the fluid pump device is removed from the vial transfer station, the adhesive cover is removed from the adhesive and the adhesive cover remains attached to the vial transfer station.

Some aspects of this aspect of the disclosure may include one or more of the following. Wherein the fluid pump device further comprising a drive motor, wherein when the drive motor rotates in a first direction, the drive motor actuates filling a reservoir with a drug for delivery, and wherein when the drive motor rotates in a second direction, the drive motor actuates infusion the drug for delivery between the reservoir and a cannula. Wherein the fluid pumping system further includes an automatic cannula inserter including: an inserter cam; a torsion inserter spring; a release lever; and a link arm, wherein the release lever prevents the drive motor from rotating in the second direction. Wherein the fluid pumping system includes wherein when the automatic cannula inserter is triggered, the release lever allows the drive motor to rotate in the first direction. Wherein the fluid pump device comprising a reflective object sensor that sends signals to a user interface. Wherein when the reflective object sensor sends a signal to the user interface indicating the fluid pump device is in contact with human skin, the user interface sends a signal to trigger the automatic cannula inserter.

Another aspect of the present disclosure includes a fluid pump device including a filling state and an infusion state; a drive motor; a crankshaft connected to the drive motor, the drive motor for rotating the crankshaft; a pump tubing; and a peristaltic pump including: a plurality of pump fingers, wherein one of the plurality of pump fingers exerts force on the pump tubing at all times once the infusion state is initiated.

Some aspects of this aspect of the disclosure may include one or more of the following. Wherein the plurality of pump fingers comprising a triangular shape located at a point of contact with the pump tubing. Wherein the pump device further includes a user interface in communication with the fluid pump device. Wherein the user interface comprising a pre-programmed pump rate of infusion. Wherein the pump device further includes a pump cover comprising an outside and an underside, wherein a reservoir is attached to the underside of the pump cover. Wherein the reservoir is pre-filled with a drug for delivery by the fluid pump device. Wherein the pump device further includes a pump base including: an automatic cannula inserter comprising: an inserter cam; a torsion inserter spring; a release lever; and a link arm, wherein the release lever prevents the drive motor from rotating in the second direction.

Another aspect of the present disclosure includes a fluid pumping system including a fluid pump device including: a pump housing; an adhesive located on the outside of the pump housing; an adhesive cover attached to the adhesive; a reflective object sensor; and an automatic inserter; a vial transfer station, wherein the adhesive cover is attached to the vial transfer station; wherein when the fluid pump device is removed from the vial transfer station, the adhesive cover is removed from the adhesive and the adhesive cover remains attached to the vial transfer station; and a user interface in remote communication with the fluid pump device.

Some aspects of this aspect of the disclosure may include one or more of the following. Wherein when the user interface receives a signal from the reflective object sensor that the pump housing is attached to human skin, the automatic inserter is triggered. Wherein the fluid pump device comprising a filling state and an infusion state. Wherein the vial transfer station comprising a station ID, wherein the user interface receives the station ID prior to initiation of the fluid pump device filling state. wherein the fluid pump device further comprising a drive motor, wherein when the drive motor rotates in a first direction, the drive motor actuates filling a reservoir with a drug for delivery, and wherein when the drive motor rotates in a second direction, the drive motor actuates infusion the drug for delivery between the reservoir and a cannula. Wherein the fluid pump device further includes: an automatic cannula inserter comprising: an inserter cam; a torsion inserter spring; a release lever; and a link arm, wherein the release lever prevents the drive motor from rotating in the second direction.

Another aspect of the present disclosure includes a system for fluid delivery including a fluid pump device; a vial transfer station; a user interface; a drug for delivery; and a fluid conduit to a patient.

These aspects of the disclosure are not meant to be exclusive and other features, aspects, and advantages of the present disclosure will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the disclosure will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of the disclosure.

FIG. 1 is a diagram of one embodiment of the system;

FIG. 2 is an illustrative embodiment representing various embodiments of the fluid conduit to patient;

FIG. 3 shows one embodiment of a single use system according to the present disclosure;

FIG. 4A shows one embodiment of a vial transfer station;

FIGS. 4B-4D show various views of one embodiment of a vial transfer station and fluid pump system;

FIGS. 5A-5C are flow diagrams of one embodiment of various methods of the present disclosure;

FIG. 6 shows a vial attached to one embodiment of the vial transfer station and fluid pump system;

FIG. 6A is a cross-sectional view of a vial attached to one embodiment of the vial transfer station and fluid pump system;

FIGS. 7A-7C are various views of a fluid pump device according to one embodiment;

FIGS. 8A-8E are various view of one embodiment of the fluid pump device;

FIG. 8F is a view of various elements of the fluid pump device according to one embodiment;

FIGS. 8G-8I are various views of one embodiment of a motor according to one embodiment of the fluid pump device;

FIG. 8J is a view of one embodiment of the motor, together with one embodiment of a cannula inserter, according to one embodiment of the fluid pump device;

FIG. 8K is a bottom view of one embodiment of a pump cover according to one embodiment of a fluid pump device;

FIG. 9 is a graphical depiction of power consumption of one embodiment of a motor according to one embodiment of the fluid pump device;

FIG. 10 is a system diagram of one embodiment of the fluid delivery system disclosed herein;

FIG. 11 shows one embodiment of a single use system according to the present disclosure;

FIG. 12A shows one embodiment of a vial transfer station;

FIGS. 12B-12C show various views of one embodiment of a vial transfer station and fluid pump system;

FIG. 13 shows a vial attached to one embodiment of the vial transfer station and fluid pump system;

FIG. 13A is a cross-sectional view of a vial attached to one embodiment of the vial transfer station and fluid pump system;

FIGS. 14A-14B are various views of a fluid pump device according to one embodiment;

FIGS. 15A-17 are various view of one embodiment of the fluid pump device;

FIG. 18 is a view of various elements of the fluid pump device according to one embodiment;

FIG. 18 is a view of one embodiment of the motor, together with one embodiment of a cannula inserter, according to one embodiment of the fluid pump device;

FIGS. 19-20 are various views of one embodiment of a motor according to one embodiment of the fluid pump device;

FIGS. 20-23B are various views of a torsion spring according to one embodiment of the fluid pump device;

FIGS. 24A-24B are various views of one embodiment of a linkage inserter;

FIGS. 25A-25B are various views of one embodiment of a sleeve inserter;

FIGS. 26A-26D are various views of one embodiment of a 90-degree inserter;

FIGS. 27A-27B are various views of one embodiments of a cam wheel inserter;

FIGS. 28A-28B are various views of one embodiment of a rotating arms inserter; and

FIGS. 29A-29B are various views of one embodiment of a rotating arms manual inserter.

### DETAILED DESCRIPTION OF THE DISCLOSURE

### Definitions

For the purposes of this disclosure, the following terms may be defined as indicated:

The terms “drug”, “fluid”, and “therapeutic” are used interchangeably to denote any substance that is being provided to a patient using the fluid pump device.

The term “patient” refers to a person or animal receiving the drug, fluid and/or therapeutic.

The term “deliver” and “delivery” may refer to providing a patient with a drug/fluid/therapeutic and may refer to subcutaneous infusion, IV infusion and/or nebulization.

Any step in any method may be accomplished by the patient or a caregiver, including a healthcare provider, with the exception of the actual delivery of the drug, which is only delivered to the patient and accomplished by the fluid pump device.

A “motor” is used herein to indicate an element which imparts the movement and/or motion of something/anything.

A patient or caregiver self-administration therapy system is disclosed. Through practically painless patient body access, a patient may receive auto-programmed delivery of drugs/fluids/therapeutics in a home setting. Step-by-step guidance is given using a User Interface that may be an App on their smartphone. The device for delivering the drug therapy may be wearable with minimum interference to the patient's daily life. Remote monitoring of the patient therapy assures compliance and positive outcomes. The system includes the ability to coordinate with standard pharmaceutical protocols from fill to finish.

The system includes a fluid pump infusion system including a fluid pump device capable of delivering subcutaneous drug therapy from 2 ml-20 ml, a volume of fluid delivery that is not feasibly delivered using autoinjectors or external infusion pumps. The fluid pump device may be used on the patient's body, or connected to an IV line already established on a patient, or in conjunction with a nebulizer and a mask to provide the vaporized medication therapy. Thus, the system may be used by a patient in the home setting or in a clinical or managed care facility to avoid having to move the patient to a hospital to receive a therapy that may be given using their existing IV line.

The system UI includes intuitive method from fill to finish and the entire therapy process may be executed by a patient or at point of administration that transfers from a standard drug container e.g., vial, to the fluid pump device within less than 60 seconds. The fluid pump device is a self-contained patient access device including a positive volumetric displacement electromechanical actuation with a self-contained secondary container of a drug. A control and communication module with a CPI is able to connect to a network and be auto-programmed by remote clinicians that may prescribe and/or adjust therapy parameters.

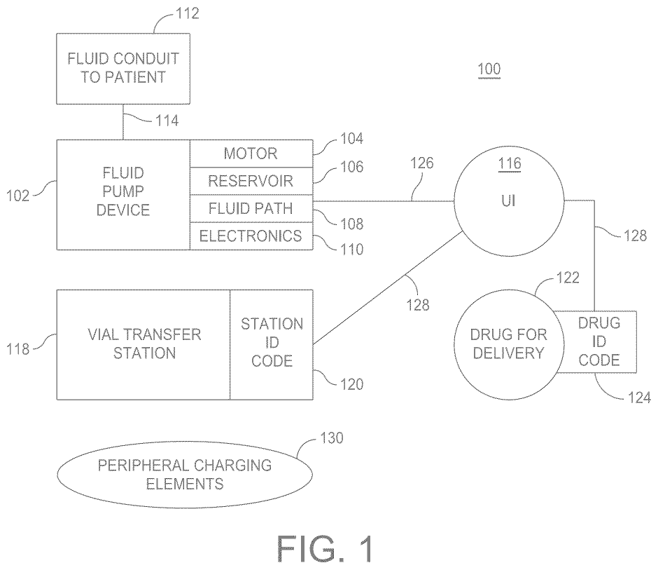

Referring to FIG. 1, one embodiment of a fluid delivery system 100 is shown. In various embodiments, the fluid delivery system 100 includes a fluid pump device 102 which, in various embodiments includes a motor 104, a reservoir 106, a fluid path 108 and electronics 110. In various embodiments, the fluid delivery system 100 includes a fluid conduit to patient 112, which is connected to the fluid pump device 102 by a fluid connection 114. The fluid delivery system 100 in various embodiments includes a User Interface (UI) 116. The fluid delivery system 100 in various embodiments includes a vial transfer station 118, which, in various embodiments, includes a station ID code 120. In various embodiments, the fluid delivery system 100 includes a drug for delivery 122 which, in various embodiments, includes a drug ID code 124. In various embodiments, a fluid pump communication link 126 is established between the fluid pump device 102 and the UI 116. In various embodiments, the fluid pump delivery system 100 includes an information link 128 between the UI 116 and the station ID code 120 and the UI 116 and the drug ID code 124. In some embodiments, one or more peripheral charging element(s) 130 may be included in the fluid delivery system 100.

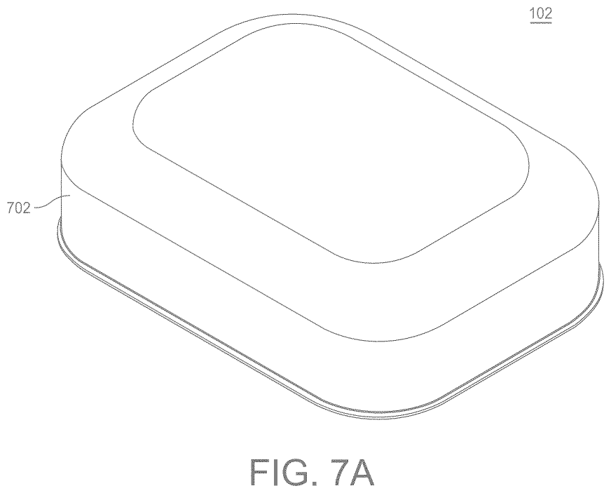

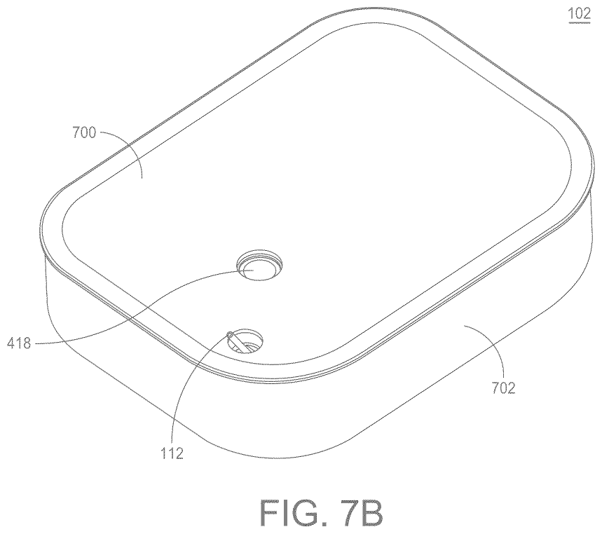

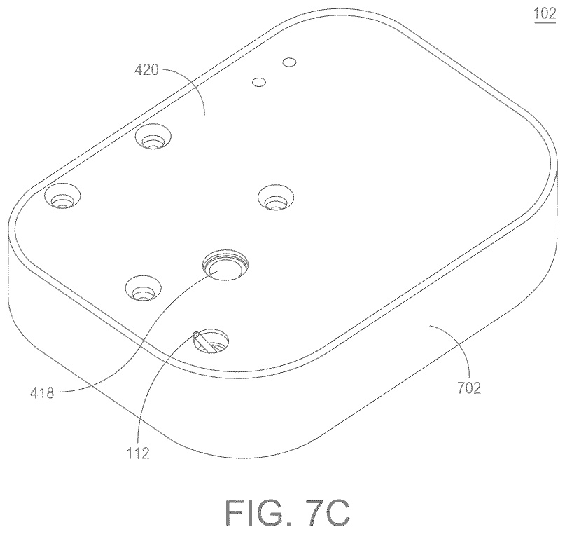

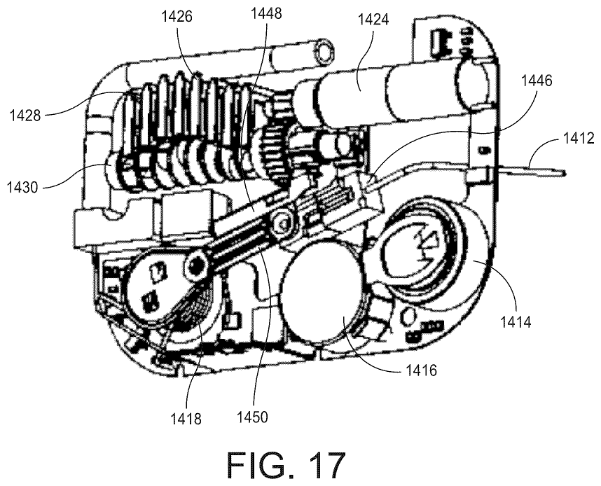

In various embodiments of the fluid delivery system 100, the fluid pump device 102 may be any fluid pump known in the art. However, in the exemplary embodiment, the fluid pump device 102 may be one of the embodiments of the fluid pump device 102 shown and described below with reference to FIGS. 7A-8K, and FIGS. 14A-29B In various embodiments, the UI 116 may be any UI known and used in the art, however, in the exemplary embodiments, the UI 116 may be a downloadable app that may be downloaded onto another electronic device, e.g., a smartphone. In other embodiments, the UI 116 may be included on a stand-alone electronic device and/or on a website accessible by a desktop/laptop computer. In some embodiments, the UI 116 may be a smart watch or other wearable electronic device, and in various embodiments, the UI 116 may be located on the fluid pump device 102. In all embodiments, the UI 116 is in communication with the fluid pump device 102 via fluid pump communication link 126. In some embodiments, the fluid pump communication link 126 may be via BLUETOOTH®, IR, NFC, or any other type of electronic communications including those known in the art.

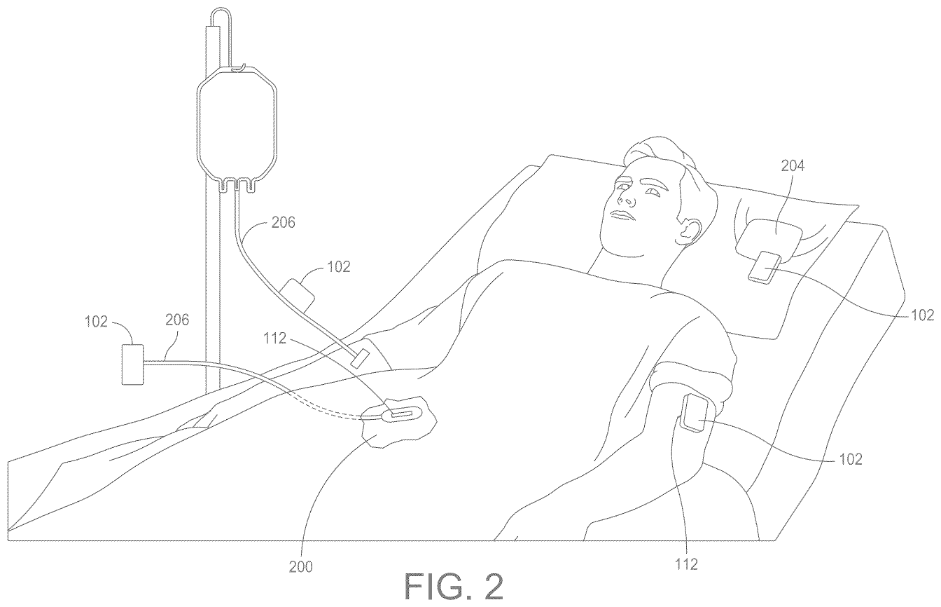

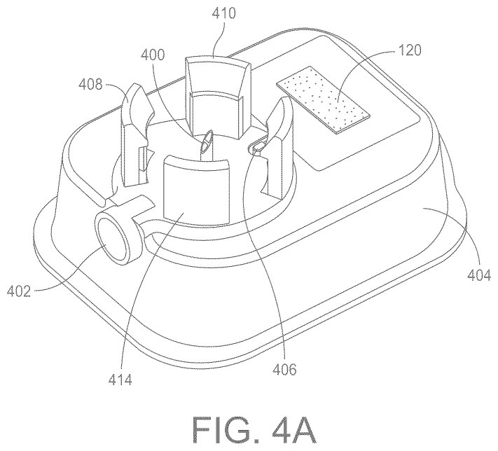

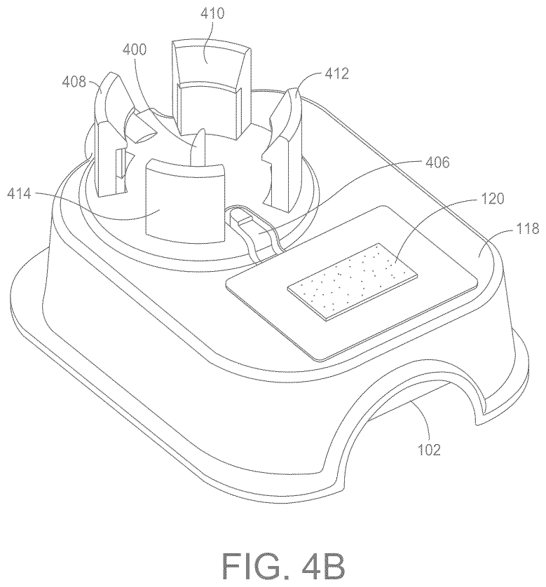

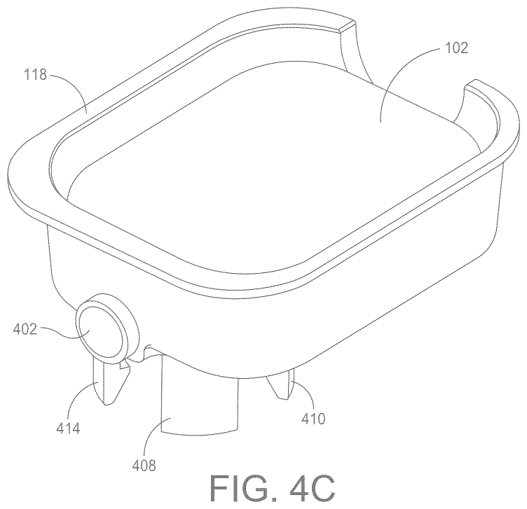

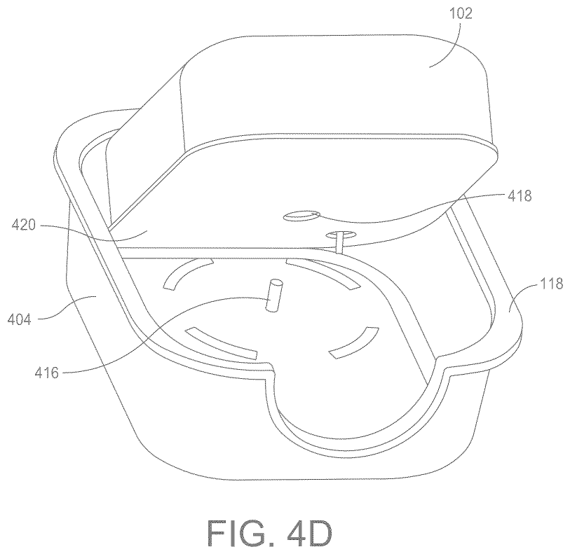

Still referring to FIG. 1, in various embodiments, the fluid conduit to patient 112 may include any conduit in which allows fluid to be pumped from the fluid pump device 102 into a patient 200, shown in FIG. 2. Referring now also to FIG. 2, a patient 200 may receive fluid pumped from the fluid pump device 102 by way of: a cannula 202 inserted into the patient 200; a mask 204 worn by the patient 200 and the fluid pump device 102 attached to the mask 204; or through an intravenous cannula 206 with the fluid pump device 102 fluidly connected to the intravenous cannula 206. In various embodiments of embodiments including a mask 204, the fluid pump device 102 is attached to a nebulizer (not shown) which can be any nebulizer known in the art. Thus, the fluid pump device 102 is attached to the nebulizer and pumps fluid into the nebulizer (not shown) which is connected to the mask 204. The patient then inhales the now inhalable fluid. In various embodiments, peripheral devices including, but not limited to, an IV port, or a nebulizer or mask may be fluidly connected to the fluid pump device 102 through the peripheral port 418 (see, e.g., FIG. 4A)

Still referring also to FIG. 2, in embodiments including an intravenous line 206, the intravenous line 206 is connected to the patient 200 by a catheter (not shown). The fluid delivery device 102 is attached to a port (not shown) that is in fluid communication with the intravenous line 206 (and also the catheter (not shown)). The fluid delivery device 102 pumps fluid into the intravenous line 206 via the port (not shown) and this flows into the patient 200 via the catheter (not shown).

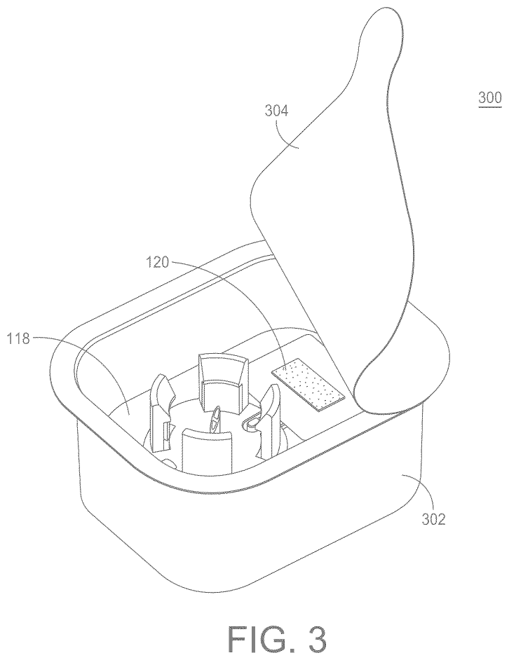

Referring now also to FIG. 3, in various embodiments, the fluid pump device 102 and the vial transfer station 118 may be packaged together in a sterile single use system 300. As described above, in various embodiments, the vial transfer station 118 includes a station ID code 120 which, in various embodiments, is a 2D barcode. However, in various embodiments, the station ID code 120 may be any identifiable indicator, including, but not limited to, an RFID tag, QR code or 3D barcode. In various embodiments, the sterile single use system 300 includes a container 302 which is sealed with a container cover 304. In various embodiments, the container cover 304 is a peel-away type of cover, but in other embodiments, may be a perforated or cut-away type of cover, or any other type of cover that may be used to seal and maintain sterility in a container.

Referring now also to FIGS. 4A-4D, once the container cover 304 is peeled away, the vial transfer station 118 may be removed from the container 302. However, in various embodiments, a patient/caregiver may choose not to remove the vial transfer station 118. In various embodiments, the vial transfer station 118 includes a spike 400, a station ID code 120, a filter 402, a holder 404, a trigger 406, claws 408, 410, 412, 414, and a needle 416. FIG. 4A shows an embodiment of the vial transfer station 118 without a fluid pump device 102 attached on the underside. However, FIGS. 4B-4D are embodiments showing the fluid pump device 102 attached to the fluid transfer station 118.

In practice, the patient/caregiver removes the container cover 304 when they are prompted to do so by the UI 116, which will occur once the patient/caregiver begins the therapy session and are ready to use the fluid pump device 102. The container cover 304 ensures that the container 302 and all of its contents, i.e., the vial transfer station 118, and the fluid pump device 102, remain sterile until and unless use is desired. Thus, in various embodiments, the UI 116 may prompt the patient/caregiver to remove the container cover 304 once confirmation of the therapy session and volume to be transferred from the drug for delivery 122 to the fluid pump device 102 is confirmed by the patient/caregiver.

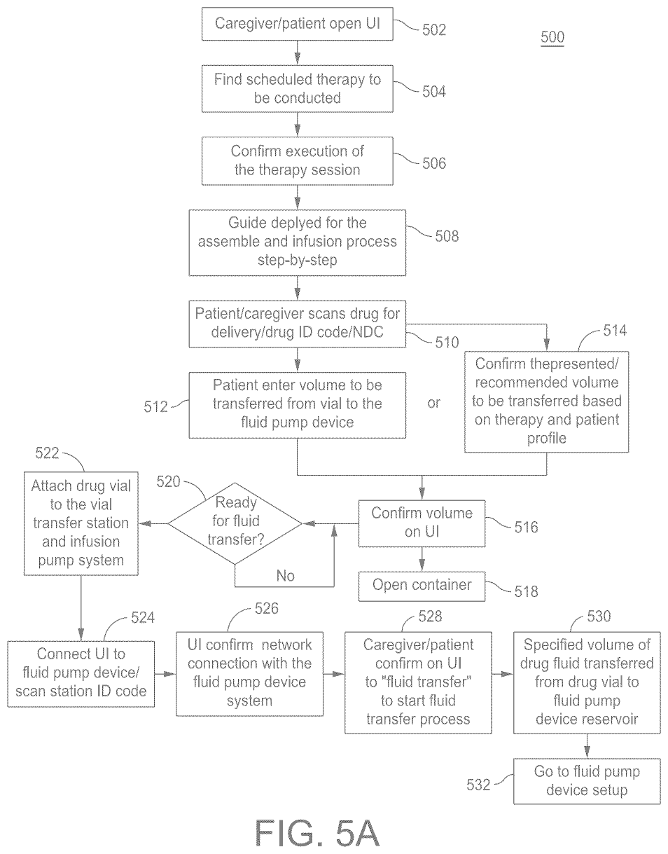

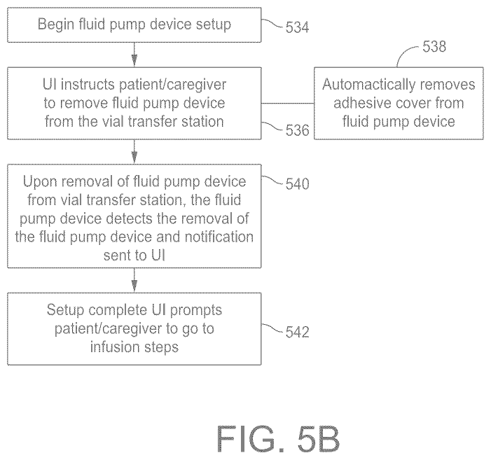

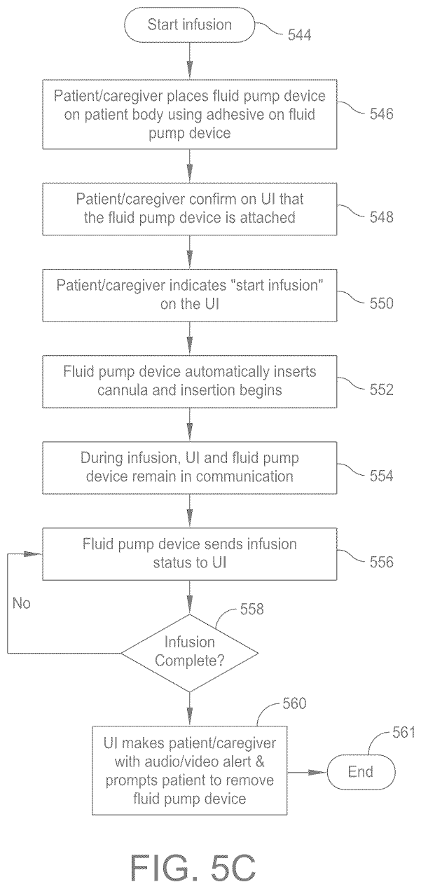

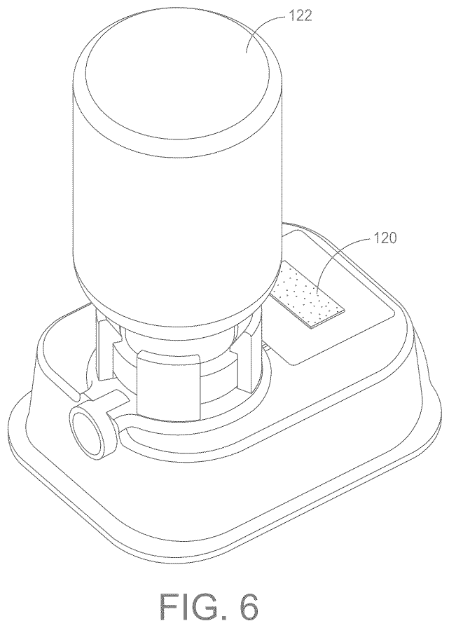

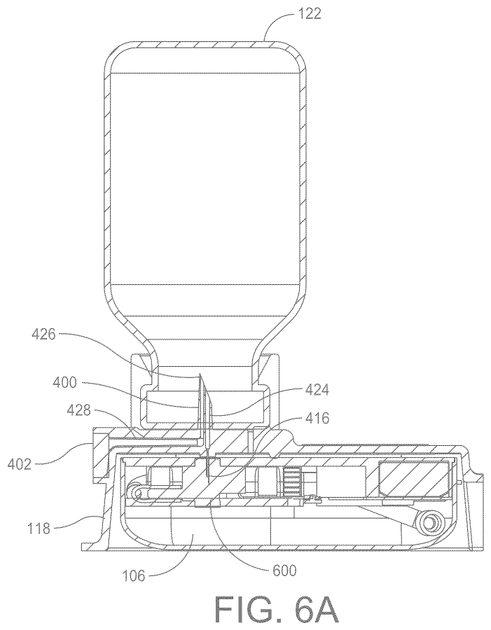

Referring now also to FIGS. 5A-6A, in various embodiments, the first step 502 in using the system 100 is the patient/caregiver opens the UI 116 and then, in the next step, navigates to/finds a scheduled therapy to be conducted 504. The patient/caregiver proceeds with following a method for fluid transfer 500 between the drug for delivery 122 and the fluid pump device 102.

In various embodiments, the UI 116 displays instructions to guide the patient/caregiver through the assemble/fluid transfer process and the infusion/fluid delivery process in a step-by-step manner. In various embodiments, the UI 116 displays both instructions and illustrations for each step to ensure the patient/caregiver follows the process correctly. This also ensures safe use of the system 100. In some embodiments, the UI 116 includes both illustrations and/or video and/or visual guidance and/or audio voice queues and/or audio alerts and/or audio notifications of the steps to further enhance the approachability of the process for the patient/caregiver. This guidance serves as readily-available instructions to the patient/caregiver.

Once authentication between the patient/caregiver and the UI 116 is successfully completed and confirmed by the system 100, the patient/caregiver confirms execution of the therapy session 506. The UI 116 then displays the guide for the particular assemble and infusion process, step-by-step, for the scheduled therapy 508.

In various embodiments, authentication may be accomplished using any method known to electronically ensure that the actual patient/caregiver is using the UI 116 and/or the fluid pump device 102 and/or the drug 122. These include, but are not limited to, one of more of the following: username/password (including two-step authentication methods) and/or biomatrix methods, e.g., face recognition. The authentication application is connected to an HTM server to authenticate the patient as user and load the corresponding patient profile onto the HTM UI 116.

The patient/caregiver next scans, using the UI 116 device, the drug for delivery 122 using the drug ID code 124 located on the drug intended for delivery to the patient. In various embodiments, the drug ID code 124 is a National Drug Code (NDC), which is assigned by the Food and Drug Administration of the United States (FDA). However, in other embodiments, the drug ID code 124 may be any code that indicates to, and is recognized by, the system 100 of which the drug is being used for the therapy. Thus, in some embodiments, the drug for delivery 122 may be a drug listed and approved by the FDA and/or other national or federal equivalent in other countries, and/or may be another type of therapeutic that is not listed or approved by the FDA and/or other national or federal equivalent, but may be prescribed by the patient's health provider.

Scanning the drug for the NDC 510 allows the system 100 UI 116 to confirm that the correct drug is being used for the intended/prescribed/scheduled therapy. This ensures safety for the patient as the system 100 will not allow the patient/caregiver to transfer a volume of an unidentified fluid into the fluid pump device 102 and therefore, prevents a mistake or unintentional delivery of a therapeutic that is not appropriate or prescribed for the intended/schedule therapy session.

Still referring to FIGS. 1-6A, upon successful scan of the drug ID code 124 by the UI 116, the UI 116 prompts the patient/caregiver to continue to the next step 512 of entering/confirming the volume of the drug for delivery 512, 514 to be transferred from the drug for delivery 122 to the fluid pump device 100. In various embodiments, and as discussed above, the drug for delivery 122 may be any drug or other therapeutic in which it is desired that it be delivered to a patient. In various embodiments, the container in which the drug for delivery 122 is stored may vary. In the exemplary embodiment described herein, the drug for delivery 122 is contained in a vial. A vial may be any size known in the art, and is a glass container which is sealed by a septum. However, in various other embodiments, any other container for the drug/fluid/therapeutic for delivery 122 may be used. These include, but are not limited to: blister packs or a container with a reconstituted drug, which may be in powder form, i.e., when shipped and/or provided to the patient/caregiver, and then, in some embodiments, there may be instruction for the patient/caregiver to reconstitute the powder, e.g., by injecting sterile saline into the container with the powder and shaking the container. This is merely an example of other embodiments of containers that may contain the drug for delivery 122, and meant to convey the breadth of the intended scope of this disclosure. For purposes of this illustrative embodiment and for purposes of providing an exemplary embodiment in this disclosure, the drug for delivery 122 is stored in a vial.

In some embodiments, the UI 116 may prompt the patient/caregiver to enter the volume to be transferred from the vial to the fluid pump device 512. In these embodiments, there may be a decision made by the patient/caregiver of the volume needed for therapy. However, in other embodiments, the UI 116 and the schedule therapy to be conducted may include a prescribed volume. In these embodiments, the prescribed volume is indicated on the UI 116 to the patient/caregiver and the patient/caregiver confirms the prescribed/recommend volume to be transferred from the vial 122 to the fluid pump device 102 based on a schedules/pre-programmed therapy and patient profile 514.

In some embodiments, once the patient/caregiver either enters the volume to be transferred from the vial to the fluid pump device 512 or confirms the prescribed/recommended volume to be transferred based on the therapy and patient profile 514, the UI 116 will display the intended volume again and request/require the patient/caregiver confirms the volume on the UI 516. In various embodiments this is required to ensure the correct volume has been selected and gives the patient/caregiver one additional chance to confirm the volume of the drug for delivery 122/from the vial 122 to be transferred to the fluid pump device 102. In various embodiments, this additional step may be beneficial/desirable for many reasons, including, but not limited to, ensuring the patient/caregiver does not make a mistake with the volume being transferred.

In various embodiments, the UI 116 may now prompt the patient/caregiver to open the container 518. In various embodiments, this is accomplished by peeling the container cover 304 off of the container 302. An exemplary embodiment of this step is shown in FIG. 3. Upon completion of this step 518, the patient/caregiver is prompted by the UI 116 and asked whether they are ready to proceed to the transfer step 520.

Once the patient/caregiver confirms they are ready for the fluid transfer to begin 520, the patient/caregiver is prompted by the UI 116 to attach the drug vial/drug container 122 onto the vial transfer station 118 in step 522. Referring now also to FIGS. 5A-5B, once the drug vial 122 is attached to the vial transfer station 118 the UI 116 prompts the patient/caregiver to proceed to connecting the UI 116 to the vial transfer and fluid pump system 422 in step 524.

Referring back to step 522, in embodiments where the drug for delivery 122 is contained in a vial, the patient/caregiver attaches the vial 122 to the vial transfer station and fluid pump system 422 by pressing the septum end of the vial 122 onto the spike 400 on the vial transfer station 118. This action forces the spike 400 of the vial transfer station 118 to pierce the septum (self-sealing membrane) of the vial 122. In various embodiments, a switch is also activated by the vial when it is inserted into the vial transfer station and fluid pump system 422. In various embodiments, after the fluid pump device 102 is removed from the vial transfer station and fluid pump system 422, that switch is disengaged. The switch, in various embodiments, turns the fluid pump device 102 on and into a filling mode. In some embodiments, a touch switch may be used.

Once the spike 400 is in fluid communication with the contents of the vial 122, this establishes a fluid path between the fluid pump device 102 and the vial 122. Additionally, with the same action, the vial 122 actuates a trigger 406 on the vial transfer station 118, which presses a switch (not shown) through the trigger 406. The switch turns on the fluid pump device 102 for filling purposes only.

The UI 116 then confirms with the patient/caregiver that the vial 122 is attached to the vial transfer station 118. In various embodiments, this may be through a prompt on the UI 116 that asks, e.g., “is the vial connected to the vial transfer station?”. The patient/caregiver needs to confirm “yes” for the process to continue.

The vial transfer station 118 includes claws 408, 410, 412, 414 which receive the vial 122 and compression hold the vial 122 in place on the vial transfer station 118. In some embodiments, there are four claws 408, 410, 412, 414, as shown in the exemplary embodiment, however, in other embodiments, there may be more or less than four claws. In various embodiments, the claws 408, 410, 412, 414 spring back to receive the vial 122, and then when the vial 122 is removed, they spring back again to allow the removal of the vial 122 from the vial transfer station 118.

Once the patient/caregiver confirms that the vial 122 is attached to the vial transfer station 118, the UI 116 prompts the patient/caregiver to connect the UI 116 to the fluid pump device 102. The patient/caregiver then connects the UI 116 to the fluid pump device 102 by scanning the station ID code 120 in step 524. Next, in step 526, the UI 116 confirms there is a network connection between the fluid pump device 102 and the UI 116. In some embodiments, the system confirms there is a BLUETOOTH® connection with the fluid pump device 102 and the UI 116.

The UI 116 then prompts the patient/caregiver to confirm on the UI 116 that the fluid transfer process should begin 528. In some embodiments, the UI 116 prompts the patient/caregiver to press a “fluid transfer” button on the UI 116 to confirm that the fluid transfer process should begin. Once the patient/caregiver confirms that the fluid transfer process should begin 528, in step 530, the specified volume of drug fluid (from steps 512/514) is transferred from the drug vial 122 to the fluid pump device 102 reservoir 106. Once the fluid transfer process is complete, in some embodiments, the UI 116 may prompt the patient/caregiver to proceed to setting up the fluid pump device 102 for the scheduled therapy to begin 532. In other embodiments, the UI 116 may automatically proceed to the next step of setting up the fluid pump device 102. In some embodiments, the patient/caregiver may indicate by navigating to the next menu and/or by confirming on the UI 116 that they wish to proceed to the next step of fluid pump device setup.

Although in some embodiments, the commands and interaction with the UI 116 are as described above in the exemplary embodiment of the UI 116, in various other embodiments, different language and buttons may be programmed into the UI 116. Thus, other embodiments are considered included within the breadth and scope of this disclosure.

Referring now also to FIG. 6A, the fluid transfer process 530 is completed by the specified/pre-determined volume of fluid from the vial 122 being transferred to the reservoir 106 of the fluid pump device 102 through the fluid path 424, which is part of the spike 400. Another portion of the spike 400 is a spike air line 426 which is fluidly connected to the filter air path 428 that leads to the filter 402. During the fluid transfer process, air is pushed into the vial 122, and then the motor 104 turns in reverse to actuate pulling fluid from the vial 122 into the reservoir 106, through the needle 416 which enters the fluid pump device 102 via the peripheral port 418. In some embodiments, a valve (not shown) may be used rather than a needle. Once the fluid transfer process is complete, the ability of the motor 104 to run in reverse will be locked such that it will not be able to turn in the reverse direction again, or until another filling process is allowed. In some embodiments, once the fluid pump device 102 is removed from the vial transfer station 118, the fluid pump device 102 is placed into a locked down mode to prevent motor 104 reversal under any circumstances. This may be beneficial/desirable for many reasons, including, but not limited to, preventing unintentional reverse motor turning which may interrupt and/or cause a failure of infusion of the drug for delivery 122. In some embodiments, while the motor 104 is turning in reverse, the UI 116 may indicate this to the patient/caregiver. In some embodiments, this indication may be an alert and/or an alarm that may include, but is not limited to, one or more of the following: visual and/or audio indications of a system failure; blinking lights; loud alarms; increasing alarms which increase in intensity the longer the alarm is sounding; vibration motor which may increase in pulse frequency until, for example, the vibration continues consistently; and/or audio indication using a voice audio that a failure or other is occurring. In some embodiments, a system failure may occur and force the fluid pump device 102 to a failure mode, not allowing further use of that particular fluid pump device 102. This may be beneficial/desirable for many reasons, including, but not limited to, preventing patient injury.

In various embodiments, during the fluid transfer process the fluid path 424 is in fluid communication with the reservoir 106 through the reservoir port 600. In various embodiments, the reservoir port 600 is a septum (e.g., a self-sealing membrane). In various embodiments, the needle 416 pierces the peripheral port 418 on the fluid pump device 102.

The fluid transfer process, in various embodiments, occurs through a low-resistance, higher-volume fluid path. This allows for a higher volume of drug/fluid/therapeutic to be filled in a short amount of time. In various embodiments, the fluid transfer process may fill at a rate of 200 ml/hour and in general, fill 5cc in two (2) minutes.

Referring now also to FIGS. 4B and 4C, in various embodiments, the fluid pump device 102 is held by the vial transfer station 118 by the holder 404 portion of the vial transfer station 118. The fluid pump device 102 is held by the holder 404 before the vial transfer station and fluid pump system 422 is put into use, during the fluid transfer process, and until the fluid pump device 102 is removed from the holder 404 portion of the vial transfer station 118.

Referring now also to FIGS. 4D and 5B, in some embodiments, the fluid pump device 102 setup begins 534 when the fluid transfer process shown and described above with respect to FIG. 5A, is complete. In step 536, the UI 116 instructs the patient/caregiver to remove the fluid pump device 102 from the vial transfer station 118. In some embodiments, the UI 116 may instruct the patient/caregiver to flip the vial transfer station and pump system upside down, so the vial 122 is sitting on a flat surface. In step 540, upon removal of the fluid pump device 102 from the vial transfer station 118, the fluid pump device 102 detects that it has been removed from the vial transfer station 118 and notification of this is sent to the UI 116. The setup of the fluid pump device 102 is now complete 542 and the UI 116 prompts the patient/caregiver to go to the infusion steps.

In various embodiments, the UI 116 detects that the fluid pump device 102 has been removed from the vial transfer station 118 using an optical sensor facing the contact surface of the fluid pump device 102. That sensor may not only sense that the pump is still attached to the vial transfer station and fluid pump system 422, but also detect if the fluid pump device 102 is placed onto the patient body.

Referring now also to FIGS. 7A-7C, in some embodiments, the fluid pump device 102 includes an adhesive 420 designed for adhering to human skin. Before and until the fluid pump device 102 is placed on the skin of a patient, the adhesive 420 is protected by an adhesive cover 700. In various embodiments, removing the fluid pump device 102 from the vial transfer station 118 automatically 538 removes the adhesive cover 700 from the fluid pump device 102. However, in some embodiments, the adhesive cover 700 may remain on the adhesive 420 until and unless a patient/caregiver removes the adhesive cover 700. In some embodiments where the adhesive cover 700 is automatically removed from the adhesive 420, the adhesive cover 700 may be attached to the vial transfer station 118 such that lifting the fluid pump device 102 out of the holder 404 automatically removes the adhesive cover 700 from the adhesive 420 of the fluid pump device 102.

Although in some embodiments, including the exemplary embodiment described herein, the fluid pump device 102 may deliver drug 122 to the patient while being attached to the patient's skin using the adhesive 420, in other embodiments, the fluid delivery device 102 is not attached to the patient's skin. In some embodiments, the fluid pump device 102 may deliver the drug 122 while being held by a band/holder that maintains the fluid pump device 102 against the skin of the patient, but while the fluid pump device 102 is not adhered to the skin.

As discussed in detail above, in various embodiments, drug 122 is delivered using the fluid pump device 102 in conjunction with an IV line 206 and/or a nebulizer and mask 204. In these embodiments, additional steps are performed to attach the fluid pump device to the IV line 206 via a port (not shown) and the nebulizer via a connection (not shown). However, in many embodiments, a similar method for starting infusion/delivery is used.

Referring now also to FIG. 5C, in various embodiments, to start infusion of the drug for delivery 122, the patient/caregiver places the fluid pump device 102 on the patient body using the adhesive 420 on the fluid pump device 102, or, in some embodiments, by attaching the fluid pump device 102 to a holder and placing the holder onto the patient's body in step 546. As discussed above, in some embodiments, this step 546 includes the patient/caregiver connecting the fluid pump device 102 to an IV port to an IV line 206 that is attached to a patient, or attaching the fluid pump device 102 to a nebulizer (not shown) connected to a mask 204 or other that is then placed onto the patient. Whichever method of connection to the patient that is used, once the connection is completed, the patient/caregiver confirms on the UI 116 that the fluid pump device 102 is attached to the patient. In various embodiments, as with all the steps disclosed herein, in various embodiments the UI 116 provides video and/or audio and/or visual guidance to the patient/caregiver for placing the fluid pump device 102 in the appropriate manner such that infusion/drug delivery may commence 546.

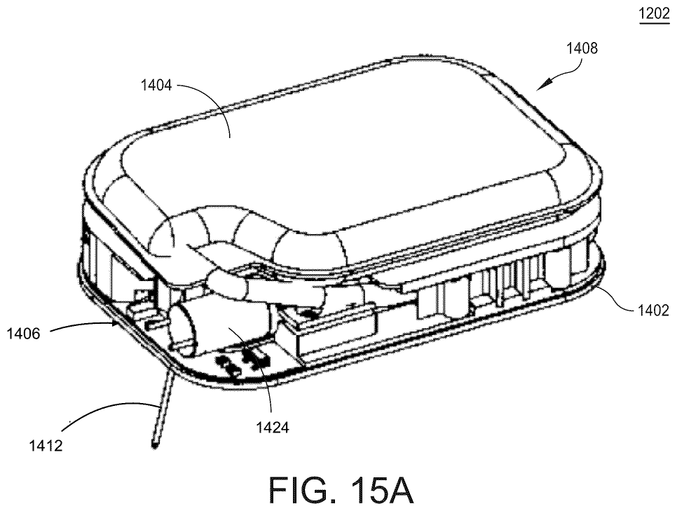

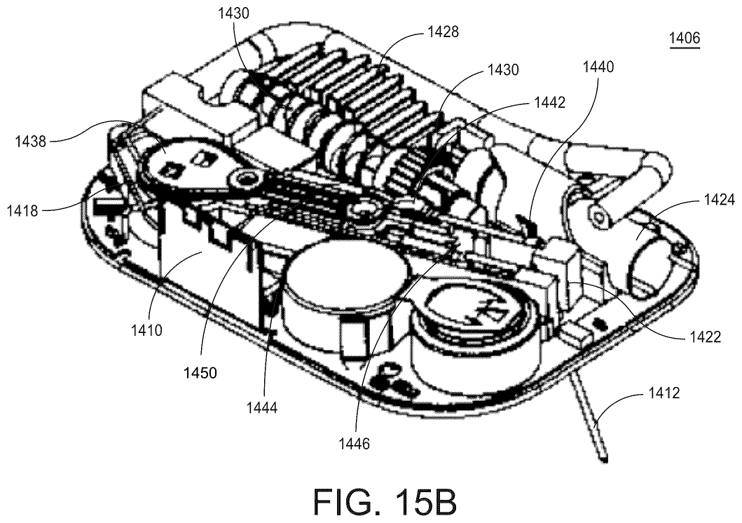

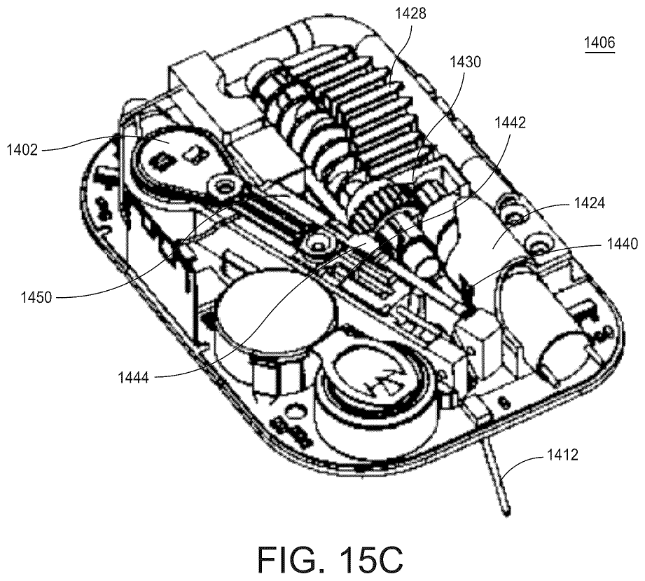

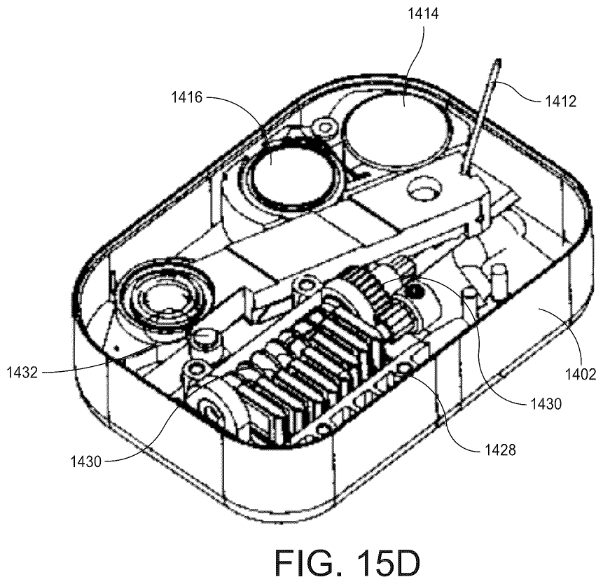

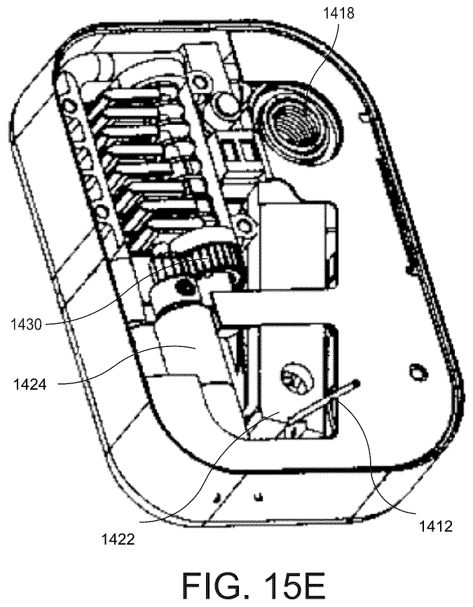

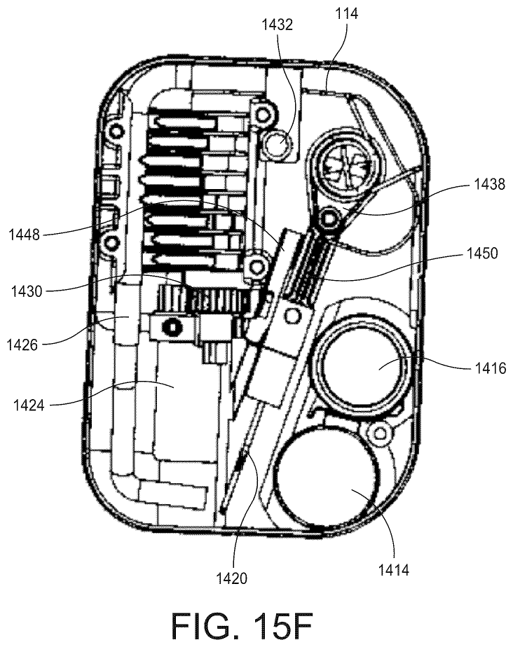

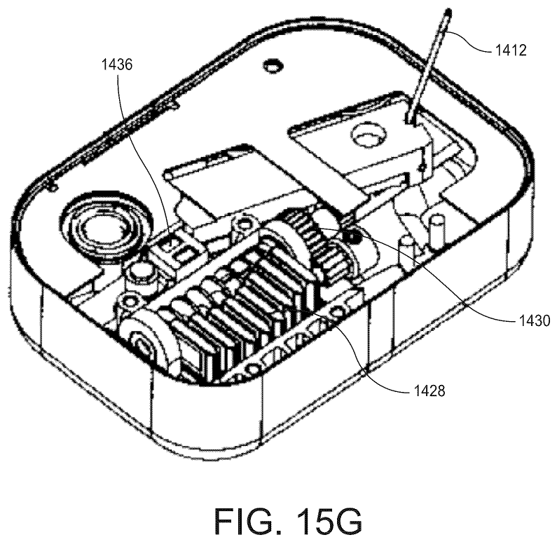

Once the fluid pump device 102 is appropriately connected to the patient, the patient/caregiver confirms this with the UI 116 in step 548. In some embodiments, the UI 116 prompts the patient/caregiver, asking “start infusion?” or words to that effect. The patent/caregiver indicates to the UI 116 to “start infusion” and/or “start delivery” 550. In some embodiments, where the fluid pump device 102 is attached to the skin of the patient, the fluid pump device 102 may automatically insert a cannula into the patient 552. In some embodiments, a Reflective Object Sensor, located within the fluid pump device 1202, is used to determine whether the fluid pump device 102 is attached to the skin. In various embodiments, this Reflective Object Sensor may also detect when a drug for delivery 122 is attached to the vial transfer station 118. This is shown and described in more detail below with reference to FIG. 15G. In some embodiments, the patient/caregiver manually inserts the cannula. However, in all embodiments, once a fluid line has been established between the reservoir 104 in the fluid pump device 102 and the patient, infusion may begin 552.

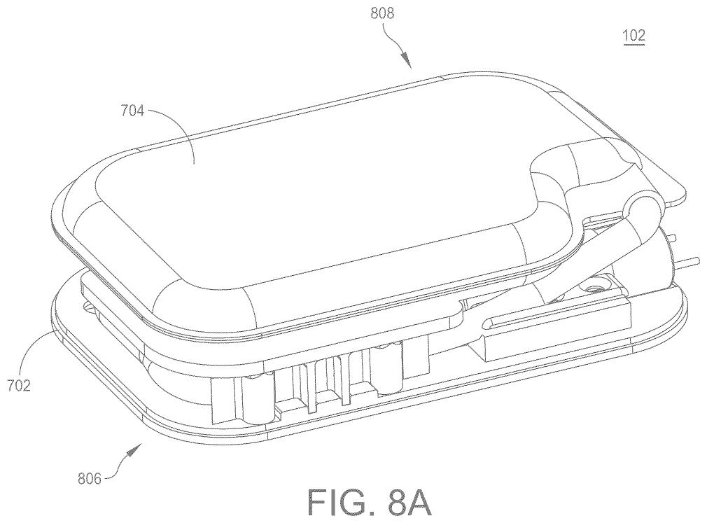

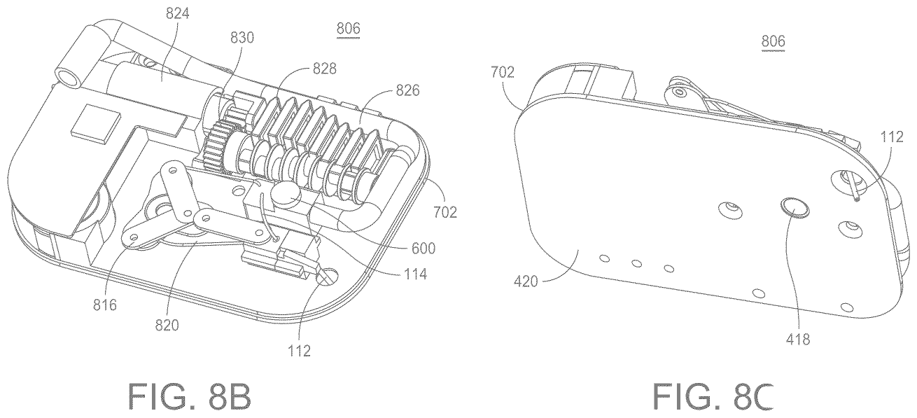

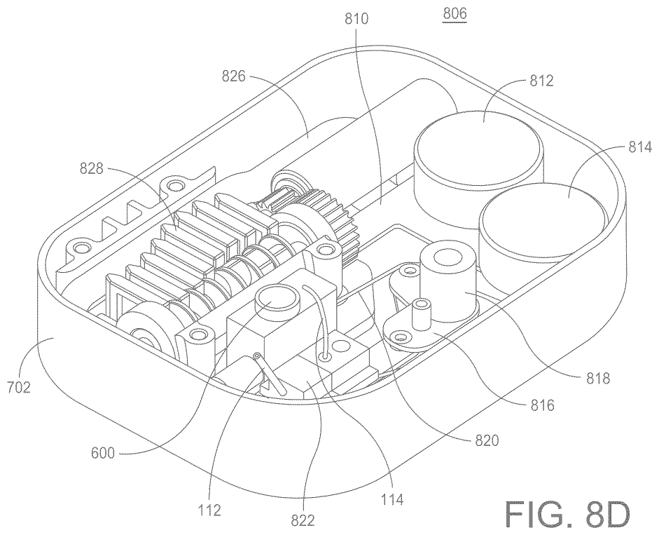

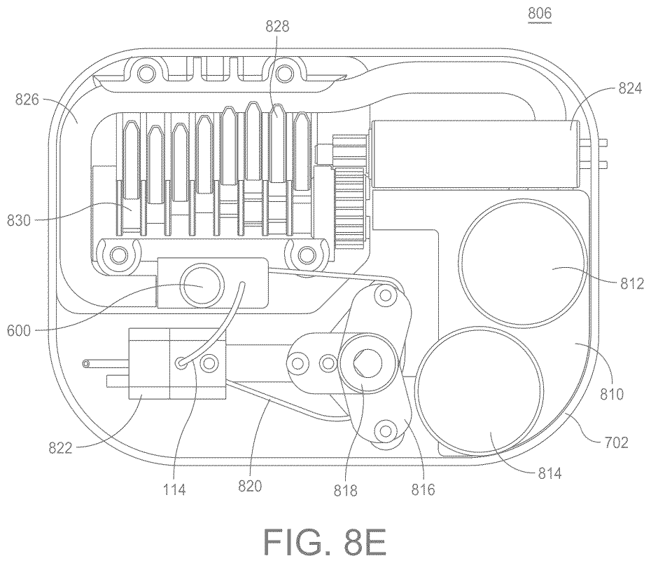

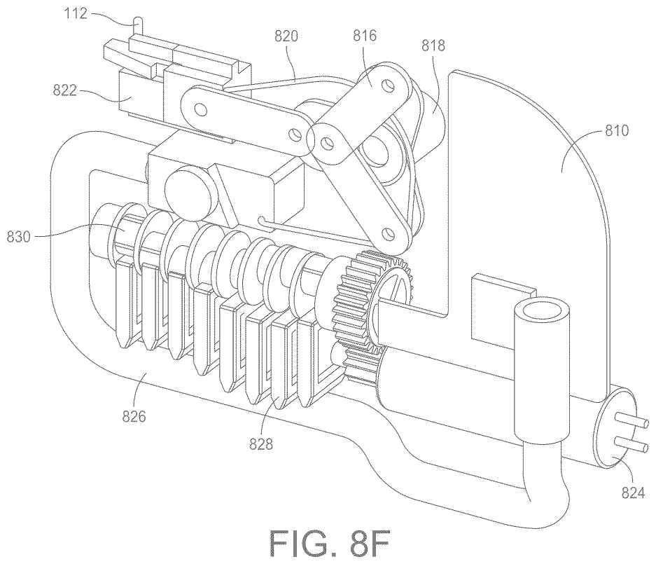

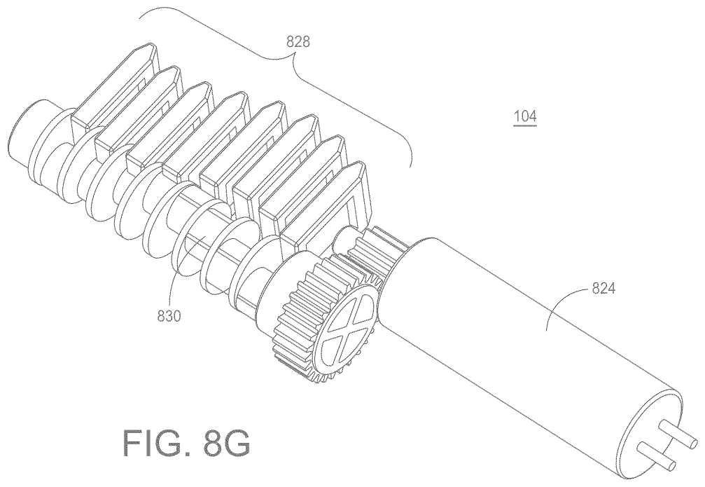

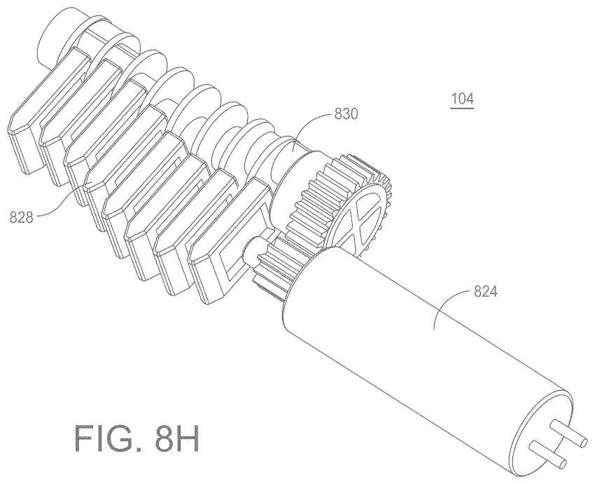

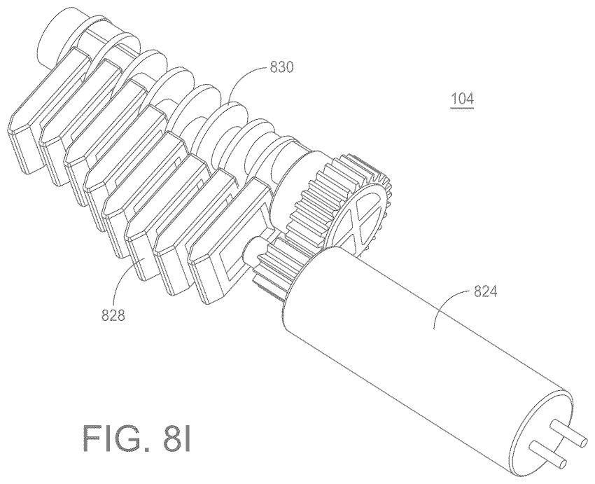

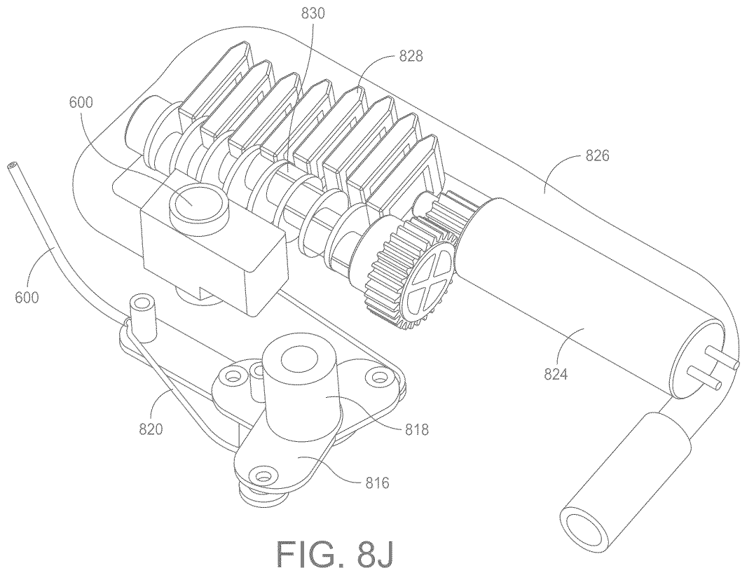

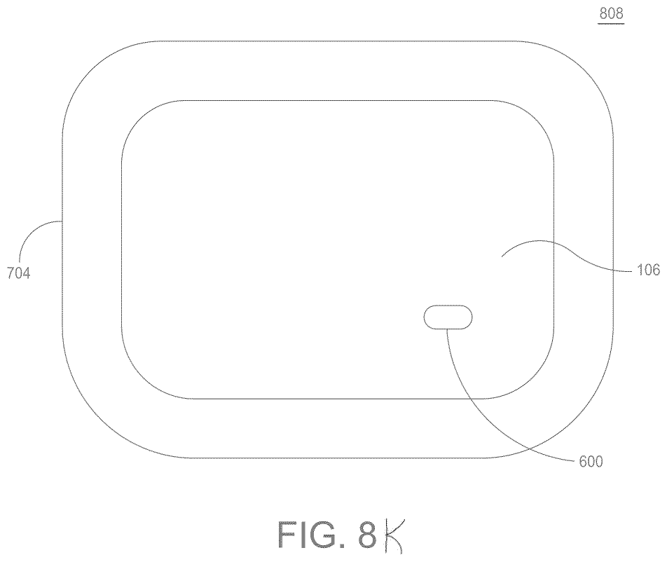

In various embodiments, during infusion, the UI 116 and the fluid pump device 102 remain in communication during the entire infusion/delivery process 554. In various embodiments, during the entire duration of infusion/delivery, the fluid pump device 102 sends infusion/delivery status updates to the UI 116 in step 556. These updates include, but are not limited to: occlusion sensing data and/or rotation of the peristaltic pump (104, see e.g., FIGS. 8F-8J) counts, and in various embodiments, this data may be used to calculate the volume of drug/fluid/therapeutic that has been delivered/infused to the patient). In step 560, once the infusion/delivery is complete 558, the UI 116 notifies the patient/caregiver with an audio and/or visual alert that it is appropriate/safe to remove the fluid pump device 102. The patient/caregiver, in various embodiments, must confirm that the infusion/delivery is complete. In other embodiments, the patient/caregiver is not required to acknowledge that the infusion/delivery is completed after being alerted. This ends 562 the therapy session.

In some embodiments, the UI 116 processes information regarding the volume of drug/fluid/therapeutic pumped to the patient to track the status of the infusion therapy. In some embodiments, the UI 116 may determine that the preprogrammed/requested volume of drug/fluid/therapeutic has not yet been delivered, even though the reservoir 106 may be empty. In some embodiments, this may trigger an alert/alarm to notify the patient and/or to notify the caregiver/healthcare provider. In some embodiments, the infusion therapy session may continue into the UI 116 determines that the total volume has been delivered.

In various embodiments, infusion is done over a high resistance flow path. In some embodiments, the infusion rate may be about 120 ml/hour. As discussed in more detail below, the infusion rate may vary depending on the embodiments of the peristaltic pump 104 used, as well as other factors. In various embodiments, the therapy prescription may include a pre-programmed rate of infusion. This may be beneficial/desirable for many reasons, including, but not limited to, one of more of the following: absorption rate of various drugs/fluid/therapeutics; and/or mitigation of pain or other sensations that may be felt by the patient during infusion and/or delivery.

Fluid Pump Device

In various embodiments, the fluid pump device 102 used in the fluid dispensing system may be any fluid pump device known in the art. In some embodiments, including the exemplary embodiments described herein, the fluid pump device 102 is a disposable patch-pump device. However, in other embodiments, other pump devices may be used including syringe pumps, peristaltic pumps and/or membrane pumps. In some embodiments, the fluid pump device 102 may be a reusable pump. These embodiments are discussed in more detail below.

Referring now also to FIGS. 7A-7C together with FIGS. 8A-8K, and FIGS. 14A-29B, exemplary embodiments of the fluid pump device 102 are shown. In various embodiments, the fluid pump device 102 is a disposable fluid pump device, and has exemplary dimensions of about 49.6×37.56×13.6 mm. However, in various other embodiments, the dimensions may vary. In embodiments where an adhesive 420 is included, the dimensions may be about 50×38×14.14 mm. In some embodiments of the vial transfer station and fluid pump system 422, the dimension may be about 60×47×31.2 mm.

In various embodiments of the fluid pump device 102 the pump outside casing or pump housing 702 may be pliable and/or soft such that it conforms to a human body. In other embodiments, part of the pump housing 702 may be pliable and/or soft while the remaining areas of the pump housing 702 may be hard/non or less-pliable. In still other embodiments, the fluid pump device 102 pump housing 702 may be hard. In various embodiments, the size of the fluid pump device 102 may be larger or smaller depending, e.g., on the intended use. In various embodiments, the intended use may require a larger reservoir. In these cases, the dimensions of the fluid pump device 102 preferably may be expanded horizontally, while the vertical profile remains consistent. In some embodiments, the fluid pump device 102 horizontal dimensions may be smaller. A smaller reservoir volume requirement allows for a smaller fluid pump device 102 length. In some embodiments where the reservoir requirements are larger, the number and/or size of the batteries may be larger, allowing ample power to deliver the larger volume of drug in the reservoir. In some embodiments, the vertical dimensions may be larger than stated herein.

Still referring also to FIGS. 7A-7C and 8A-8K, in various embodiments, the fluid pump device 102 pump housing 702 includes a reservoir housing 704 and a pump base housing 702. In some embodiments, the reservoir housing 704 may be a soft, pliable material while the pump housing 702 may also be in a soft, pliable material. However, in various embodiments, the reservoir housing 704 and/or pump housing 702 may be made from the same or different materials one from another, and/or either or both may be made from soft/pliable material and/or harder material.

In various embodiments, the reservoir 106 is attached to the underside of the reservoir housing 1404 (see FIG. 8K), together with the reservoir port 1432, make up one embodiment of the pump cover 148. In some embodiments, the reservoir 106 is a plastic bag of a predetermined size. The size of the reservoir 106 may vary depending on many factors, including, but not limited to: the drug type and/or the therapy type and/or volume of drug to be delivered in the therapy session. In various embodiments, the reservoir 106 may hold 5 ml of fluid. However, in other embodiments, the reservoir 106 may hold between 2 ml-20 ml of fluid. In various embodiments, the reservoir 106 is composed of a plastic membrane that forms a sack/bag. The reservoir 106 includes a reservoir port 1432, providing access to the needle 1216 for filling and to the fluid connection 114. However, in some embodiments, the reservoir may be pre-filled, e.g., by a pharmacist and/or by a pharmaceutical/drug company, and the reservoir 106 provided, already filled to the predetermined volume, to the patient/caregiver. In various embodiments, the reservoir 106 may be formed partially with the reservoir housing 1404, that is, the underside of the reservoir housing 1404 may serve as one side of the reservoir 106.

The reservoir port 600/1432 is also used to access the drug that was loaded into the reservoir 106 for delivery to the patient via a fluid connection 114. In some embodiments, which includes the exemplary embodiments shown and described herein, the drug is delivered from the reservoir 106 to the patient using a cannula 1412 connected to the reservoir 106 via the fluid connection 114. In various embodiments, the fluid path 108 within the fluid pump device 1202 includes the path the fluid follows between the reservoir 106 and the cannula 1412.

In various embodiments, the reservoir 106 may be constructed from any material desired. In various embodiments the reservoir 106 may be constructed from material that is compatible with the drug/therapeutic in which is intended to be contained within the reservoir 106. In some embodiments, the reservoir 106 is constructed of EVA.

In various embodiments, the fluid pump device 1202 includes electronics 110 to pump the fluid from the drug for delivery/vial 122 to the reservoir 106 and from the reservoir 106 to the patient. In some embodiments, the electronics 110 include, but are not limited to, a PCB 1410 and batteries 1414, 1416 to control and power the motor 104. In various embodiments, the PCB 1410 may be an MCU with BLUETOOTH® function. However, in other embodiments, a different PCB 1410 may be used. In the exemplary embodiments, as shown and described herein, the batteries 1414, 1416 may be LR46 batteries or any other type of battery in the art. In various embodiments, the battery size and number may be selected based on many factors, including, but not limited to: amount of fluid to pump (both in the fill stage and delivery stage); the viscosity of the fluid; and the amount of time over which the therapy session/drug delivery is predetermined to occur. In the exemplary embodiment, the batteries 1414, 1416 include enough power to pump 5 ml of drug/therapeutic to a patient and to run the fluid pump device 1202 for 3 days. However, in other embodiments, different batteries and different numbers of batteries may be selected provide the necessary power to deliver the intended therapy.

In various embodiments of the fluid pump device 1202, the fluid pump device 1202 may be reusable. In some of these embodiments, the batteries 1414, 1416 (or, in various embodiments, as discussed above, other sizes and/or a different number of battery/batteries may be used, but for the purposes of this disclosure, batteries 1414, 1416 refer to any one or more of battery/batteries in the fluid pump device 1202) may be rechargeable batteries 1414, 1416. In these embodiments, other elements may be included into the fluid delivery system 100 which may include, but are not limited to: a charging device; a charging station; and/or a charging port. All of these additional elements that may be added to the fluid delivery system 100 may collectively be referred to as peripheral charging element(s) 130 (see FIG. 1). In various embodiments, also included in the peripheral charging element(s) 130 may be a power cord and/or peripheral/external battery pack (not shown) that may power one of more devices shown and/or described with reference to one or more embodiments herein, e.g., a nebulizer and/or an IV pumping system (not shown).

In some embodiments, the fluid pump device 1202 may include an internal cannula 1412. As discussed above, in some embodiments, the cannula 1412 is automatically inserted into the patient once the fluid pump device 1202 is adhered to/attached to the patient's skin. However, in various embodiments of the fluid pump device 1202 an external cannula 1412 or other external fluid conduit to the patient, may be used. These include, but are not limited to, a cannula 1412 in fluid communication with the fluid pump device 1202 via a predetermined length of tubing 206; a port connection to an IV line 206; and/or via a nebulizer (not shown) and a mask 204.

However, in various embodiments, a cannula 1412 may be housed within the pump base 1406 and include an assembly for cannula insertion that, in some embodiments, including the exemplary embodiment shown and described herein, may include an inserter 816 powered by a spring 1418, and an inserter needle 1420 which wraps around the inserter 816 and into an adapter 822. In various embodiments, and as shown in FIG. 5C, in step 552, when the UI 116 receives confirmation from the patient/caregiver to “start infusion” in step 550, the fluid pump device 1202 automatically inserts the cannula 1412 and infusion beings. In various embodiments of this embodiment of the fluid pump device, method and system, it is in step 552 that the UI 116 sends a command to the fluid pump device 1202 PCB 1410 to trigger the inserter 816. Before this point, the spring 1418 is held in tension. When the trigger is given, the spring 1418 is released and the inserter 816 is triggered to propel the inserter needle 1420 through the adapter 822 and into the cannula 1412, and both the inserter needle 1420 and cannula 1412 are inserted into the patient. At the end of this insertion motion, the inserter spring 1418 retracts, pulling the inserter needle 1420 out of the cannula 1412 and the patient, leaving the cannula 1412 inside the patient for subcutaneous delivery.

In various embodiments, the inserter needle 1420 is made from medical-grade stainless steel. In some embodiments, the cannula 1412 is made from medical-grade plastic. In some alternate embodiments, a stainless-steel cannula 1412 may be used, replacing the plastic cannula, and in these embodiments, a separate inserter needle 1420 is not needed. Thus, in these embodiments, and in some embodiments of these embodiments, using a slightly modified inserter system as the one shown and described herein, the stainless-steel cannula may be wrapped around the inserter 816 and the inserter spring 1418 propels the cannula 1412 into the patient, but does not retract. Thus, the steel cannula remains in the patient and delivery/infusion is completed using the stainless-steel cannula. In various embodiments, the stainless-steel cannula system described herein may be preferably used only where the drug/therapeutic being delivered is of a low/lower viscosity. However, in other embodiments, a stainless-steel cannula may be used for the delivery/infusion or any fluid/drug/therapeutic.

Although one embodiment of an inserter is discussed with reference to these embodiments of the fluid pump device, other embodiments of the inserter system are disclosed and shown herein, and any one of those inserter systems may be used in conjunction with the fluid pump devices described and shown herein.

Referring now also to FIGS. 7A-7D and 8A-8K, in various embodiments of the fluid pump device 102 a motor 105 is used to pump fluid from the drug for delivery 122 (e.g. vial 122) to the reservoir 106, and from the reservoir 106 to the patient via the fluid conduit to the patient 112. An exemplary embodiment of the motor 104 used in the fluid pump device 104 includes a peristaltic pump 104. In other embodiments, the motor 104 may be any device or combination of devices that work to move fluid from the drug for delivery 122 to the reservoir 106 and from the reservoir 106 to the patient. Other embodiments of motors 104 which may be used for the fluid pump device 102 include, but are not limited to: another embodiment of a peristaltic pump; a membrane pump; a syringe pump; and/or any positive displacement pump, and/or any motor known in the art.

Still referring also to FIGS. 7A-7C and 8A-8K, the peristaltic pump 104 disclosed herein is an exemplary embodiment of the motor 104. In the exemplary embodiments, the peristaltic pump 104 includes a driver motor 824 that causes the crankshaft 830 to rotate. The crankshaft 830 interacts with the pump fingers 828 to cause the pump fingers 828 to interact and effect the pump tubing 826. The pump fingers 828 cause the fluid to be pumped from the reservoir 106 to the cannula 112 at a prescribed flow rate. The flow rate may be varied, e.g., by varying the rotational speed of the crankshaft 830.

In various embodiments, the drive motor 824 is a DC motor, and any DC motor known in the art may be used. The crankshaft 830 is designed such that it interacts with the pump fingers 828 such that the plurality of the pump fingers 828 are articulated at different moments, during the full rotation of the crankshaft 830, to exert pressure onto the pump tubing 826 and move the drug/fluid/therapeutic from the reservoir 106 to the fluid conduit to the patient 112.

In various embodiments, the pump tubing 826 is made from PVC, however, in other embodiments, the pump tubing 826 may be made from any material desired. In the exemplary embodiment, the PVC pump tubing 826 provides a high resistance and therefore, a more accurate volume of fluid/drug/therapeutic may be pumped from the reservoir 106 to the fluid conduit to patient 112 per rotation of the crankshaft 830. Additionally, the PVC pump tubing 826 resistance allows for higher viscosity fluids/drugs/therapeutics to be pumped using the fluid pump device 102. This may be beneficial/desirable for many reasons, including, but not limited to, the ability to deliver monoclonal antibody treatments and other drug therapies that include high viscosity fluids/drugs/therapeutics. As the PVC pump tubing 826 is more rigid than, e.g., a membrane or other, less rigid material, the pump tubing 826 will maintain its integrity throughout the fluid pump device 102 life/while in use. The PVC pump tubing 826 together with the peristaltic pump 104 allows for high torque pumping and high-volume pumping, i.e., pumping fluid/drug/therapeutic at 20 psi and at a rate of 200 ml/hour, or cc in less than 2 minutes.

Still referring to FIGS. 7A-7C and FIGS. 8A-8K, in various embodiments, the peristaltic pump 104 shown and described herein has many benefits in addition to those discussed above. In the exemplary embodiment, the drive motor 824 is powerful enough to pump fluid/drug/therapeutic as described above, however, is small enough that it does not require a high level of power to turn the crankshaft 830. Thus, a higher volume of fluid/drug/therapeutic may be pumped by the peristaltic pump 104 (and the fluid pump device 102) using a low amount of power. This may be beneficial/desirable for many reasons, including, but not limited to: ability to pump a higher volume of fluid/drug/therapeutic using smaller batteries/less power; allowing for longer therapies to be delivered by the fluid pump device 102 using a single charge of the batteries; requiring smaller batteries therefore allowing for a smaller footprint design for the pump housing 702 and overall footprint and size/dimensions of the fluid pump device 102. A smaller profile is beneficial/desirable for many reasons, including, but not limited to: providing a lighter/smaller pump may be more easily worn by a patient; providing a lighter/smaller pump may be more easily attached to an IV line 206 or nebulizer and mask 204 such that a patient may be ambulatory while receiving therapies delivered by the fluid pump device 102, including being ambulatory while receiving fluid/drug/therapy from the fluid pump device 102 while also connected to an IV line 206.

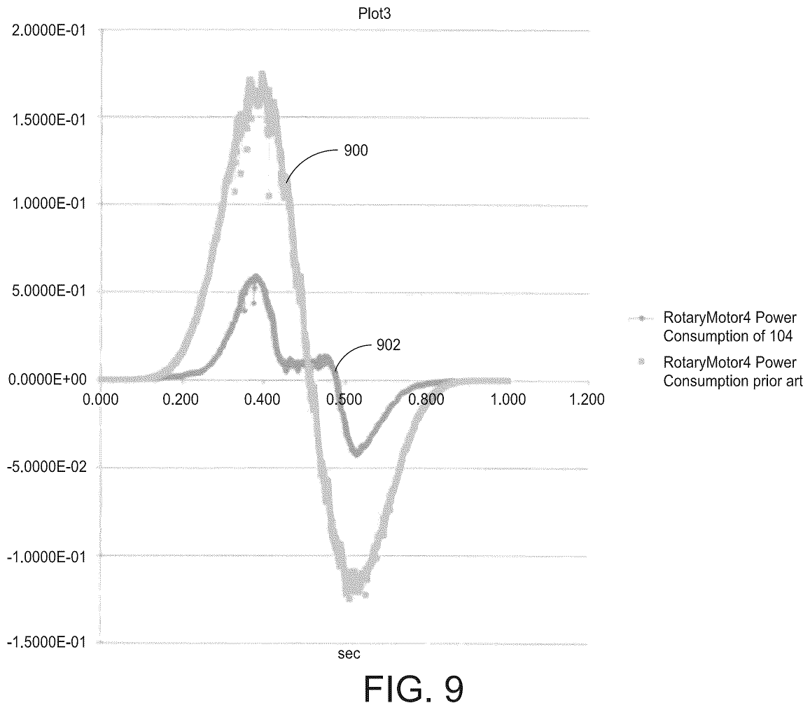

Referring now also to FIG. 9, the peristaltic pump 104 shown and described herein is an exemplary embodiment of the peristaltic pump 104 for the fluid pump device 102. This design of the exemplary peristaltic pump 104 is more power efficient compared with a prior art peristaltic pump. As may be seen from inspection, the power consumption 902 of the peristaltic pump 104 described and shown herein as the exemplary peristaltic pump 104 is substantially lower compared with the power consumption 900 of the prior art peristaltic pump 900.

Still referring also to FIGS. 7A-7C and 8A-8K, in the exemplary embodiments, the peristaltic pump 104 may be more durable and reliable for many reasons, including, but not limited to, that the peristaltic pump 104 does not include a valve. The peristaltic pump 104, in operation, always includes where one pump finger 828 is placing force on the pump tubing 826. This acts in a similar manner as a valve, however, has many advantages, including, but not limited to: performing as a safe, accurate, and reliable pump with less parts, thus may be constructed lighter and smaller than other pumps that perform similar functions, and still pump at 20 psi. In various embodiments of the peristaltic pump 104 the pump fingers 828 include a design where the point of contact on the pump tubing 826 is triangularly shaped. This provides for more pressure exertion on the pump tubing 826 as compared with a flat or rounded surface, however, does not require addition power to exert a higher force. Thus, the exemplary embodiment of the peristaltic pump 104 shown and described herein includes the ability to pump fluid/drug/therapeutic at a higher psi, e.g., 20 psi, which allows for the pumping of higher viscosity drug/fluid/therapeutic to be delivered, and at higher volumes for unit time.

The peristaltic pump 104 shown herein is an exemplary embodiment for a patch-sized fluid pump device 102. However, the design of the peristaltic pump 104 may be scaled larger or smaller depending on the type of drug/fluid/therapeutic and/or therapy being delivered using the fluid pump device 102. Thus, e.g., if larger volumes of drug/fluid/therapeutic are desired to be pumped/delivered to a patient in a short amount of time, a faster flow rate may be desired. The size of the peristaltic pump 104, including the size of the pump fingers 828, crankshaft 830, and drive motor 824 may be increased to reach a desired pumping volume/aliquot per rotation of the crankshaft 830. Conversely, the peristaltic pump 104 may be scaled down to accommodate very small delivery volumes and/or delivery of a single aliquot over, e.g., a short period of time, which may include, but is not limited to, less than 1 minute to 5 minutes. In the exemplary embodiments, the peristaltic pump 104 pumps 0.02 ml-0.03 ml per rotation.

In various embodiments of the peristaltic pump 104, there is a sensor (not shown) that counts the crankshaft 830 rotations. That same sensor provides a feedback loop to a CPU located on the PCB 810 which, in various embodiments, validates that the crankshaft 830 actually rotated, i.e., that the crankshaft 830 is functioning, after current is applied to the drive motor 824. Thus, both while transferring fluid from the vial 122 to the reservoir 106, and while delivering/infusing the fluid from the reservoir 106 to the patient via a fluid conduit to the patient 112, the UI 116 receives confirmation from the sensor (via the CPU) of the number of rotations of the crankshaft 830 that are completed. Based on this sensor data, the system, in various embodiments, may calculate the volume pumped. In various embodiments, this sensor and feedback loop allows the UI 116 to determine when the fluid transfer is complete (step 530, FIG. 5A) and when infusion is complete (steps 556, 558, FIG. 5C). Thus, the UI 116 may verify that the volume requested by the patient/by a preprogrammed therapy for infusion to the patient has been delivered, and that the volume of fluid requested for transfer from the drug for delivery 122 (e.g., vial 122) has been transferred to the reservoir 106.

In various embodiments, the peristaltic pump 104 may be beneficial/desirable for additional reasons, including, but not limited to: if air is in the pump tubing 826, the peristaltic pump 104 will still be able to pump the drug/fluid/therapeutic. Thus, air bubbles are mitigated as a consequence of the design of the motor 104 used in the exemplary embodiments of the fluid pump device 102.

In various embodiments, the fluid pump device 102, 1202 includes an occlusion sensor. In some embodiments, the occlusion sensor may be a pressure sensor or an optic sensor. The location of the occlusion sensor may be anywhere along the fluid path, including, but not limited to, where the drug/fluid/therapeutic enters the cannula 112 or other fluid conduit to the patient.

In some embodiments of embodiments including a nebulizer, an ETO2 sensor may be embedded into the fluid pump device 102. This may be beneficial/desirable for many reasons, including, but not limited to measuring the outcome of the nebulized medication delivery.

In various embodiments of the fluid delivery system 100 shown and disclosed herein, communications between the fluid pump device 102 and the UI 116 are ongoing for many reasons, including, but not limited to: for ensuring patient safety and compliance with prescribed therapy.

In various embodiments, a patient portal may be setup through the source of the drug/fluid/therapeutic, e.g., a drug company/pharmaceutical company. The UI 116 is a patient “app” that may be on any personal electronic device, e.g., a smartphone. As discussed in more detail above, the patient/caregiver uses the UI 116 to scan both the drug 122 and the vial transfer station and fluid pump system 422. This information may be shared with the patient portal and a confirmation may be made that the patient/caregiver is using not only the correct drug 122 and fluid pump device 102, but that neither is a counterfeit. Thus, scanning these elements of the system serves to ensure patient safety and that the correct therapy will be delivered to the correct patient. In various embodiments, the fluid delivery system 100 will not allow the filling steps until and unless both the drug 122 and the vial transfer station and fluid pump system 422 are authenticated.

In various embodiments, once the vial transfer station and fluid pump system 422 is scanned, this information is sent to a server to indicate that the fluid pump device 102 in that vial transfer station and fluid pump system 422 has been used. This is beneficial/desirable for many reasons, including to prevent re-use which may be dangerous to a patient; and/or to confirm compliance by the patient that the prescribed therapy has been completed.

In various embodiments, communications between the fluid pump device 102 and the UI 116 are completed via a cloud-based device gateway. Fleet management service software communicates to the communication module of the UI 116 to connect the fluid pump device. The connection to the fluid pump device 102 is separate and maintained by the conduit app/UI 116. Thus, communication to the fluid pump device 102 and the service side, e.g., authentication, of the system 100 may be accomplished.

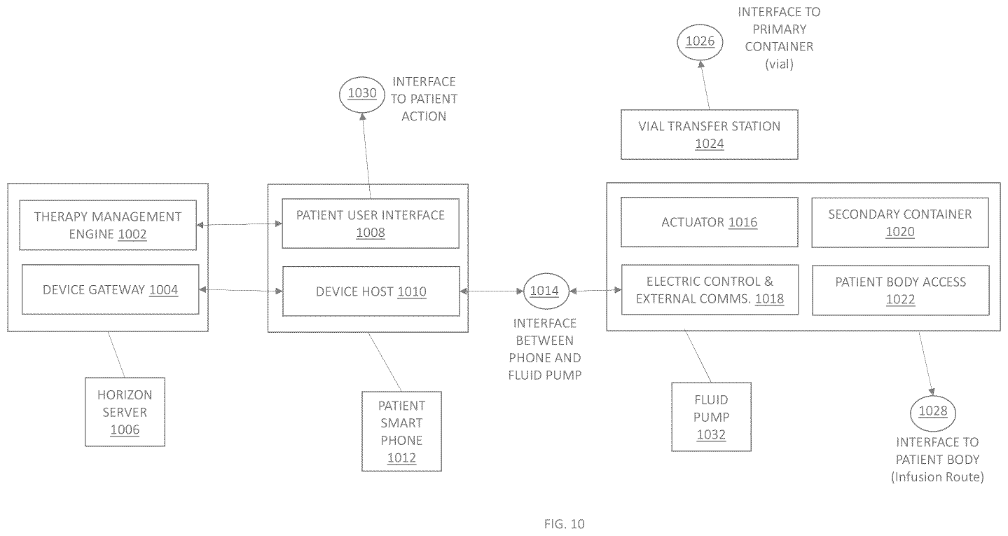

Referring now also to FIG. 10, a system diagram of one embodiment of a fluid delivery system 1000 is shown. The system 1000 includes a therapy management engine 1002, a device gateway 1004, which in various embodiments, are held on a Horizon server 1006. The system 1000 also includes a patient user interface 1008, and a device host 1010, which, in some embodiments, is located on the patient smart phone 1012. In various embodiments, the patient user interface 1008 interfaces to patient action 1030. The system 1000 in various embodiments includes an interface 1014 between the device host 1010/patient smart phone 1012 and the fluid pump phone and fluid pump. In various embodiments, the system 1000 includes an actuator 1016, electric control and external communication 1018, a secondary container 1020 and patient body access 1022. An interface to the patient body 1028 is provided to the patient body access. In various embodiments, the system 1000 includes a vial transfer station 1024 and an interface to primary container (vial) 1026 is provided to the vial transfer station 1024.

### Further Exemplary Embodiments

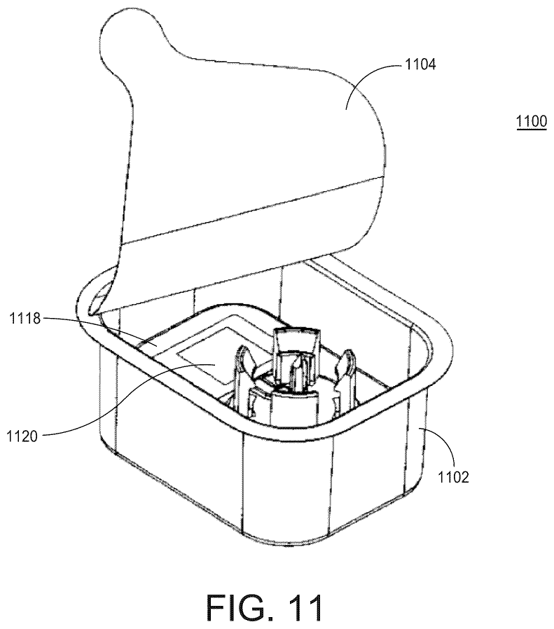

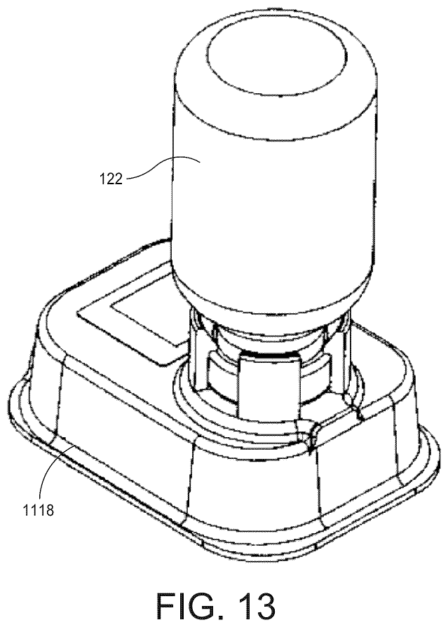

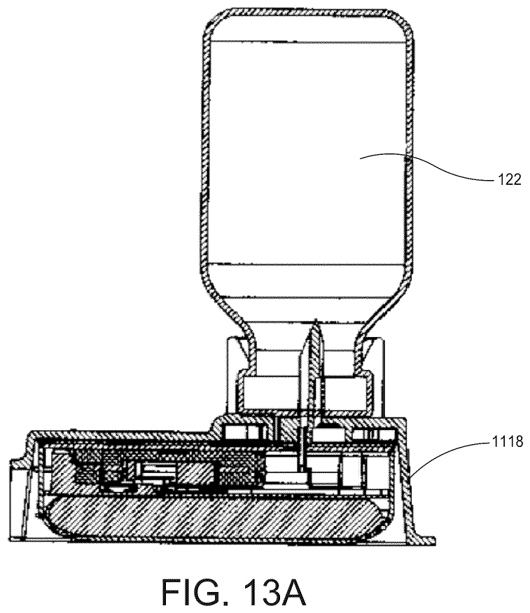

Referring again to FIGS. 3-4D and FIGS. 6-6A, and their description thereof above, in various embodiments, the fluid pump device 102 and the vial transfer station 118 may be packaged together in a sterile single use system 300. However, another embodiment of a system including a fluid pump device and vial transfer station are shown and described in FIGS. 11-13A. Referring now also to FIGS. 11-13A, the vial transfer station 1118 includes a station ID code 1120 which, in various embodiments, is a 2D barcode. However, in various embodiments, the station ID code 1120 may be any identifiable indicator, including, but not limited to, an RFID tag, QR code or 3D barcode. In various embodiments, the sterile single use system 1100 includes a container 1102 which is sealed with a container cover 1104. In various embodiments, the container cover 1104 is a peel-away type of cover, but in other embodiments, may be a perforated or cut-away type of cover, or any other type of cover that may be used to seal and maintain sterility in a container.

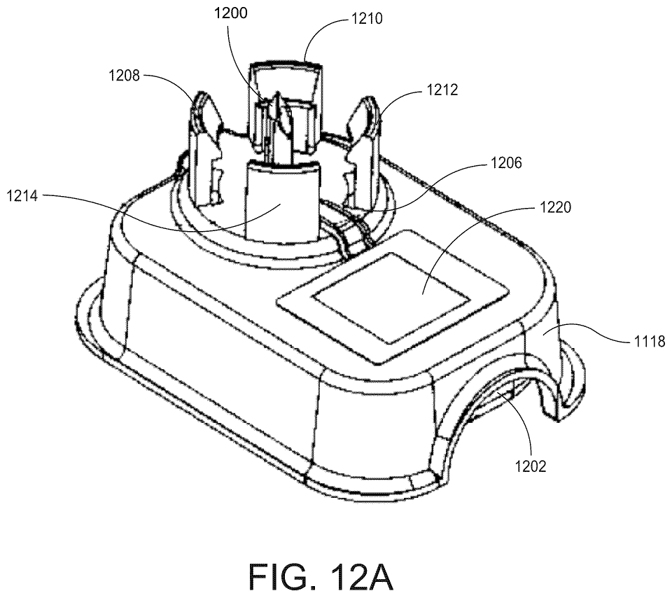

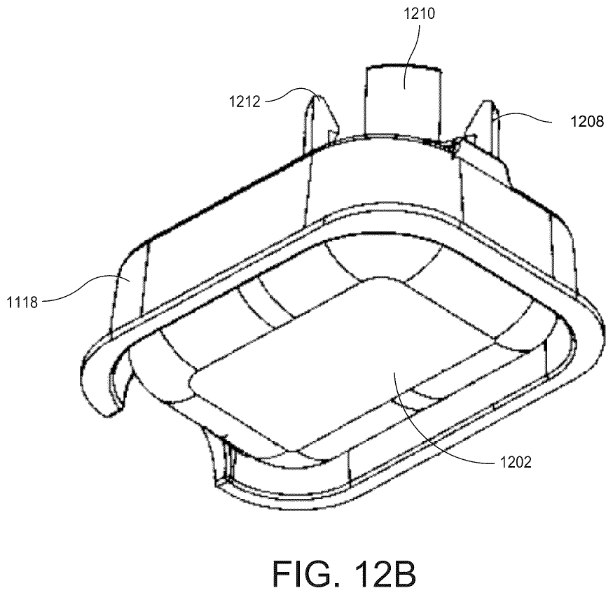

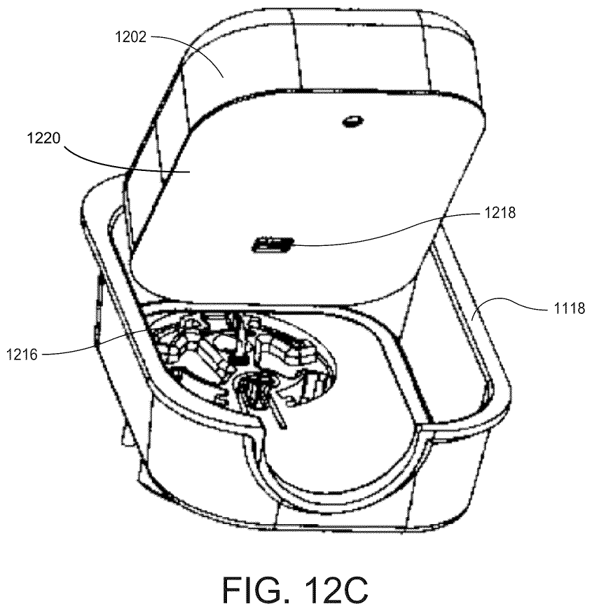

Once the container cover 1104 is peeled away, the vial transfer station 1118 may be removed from the container 1102. However, in various embodiments, a patient/caregiver may choose not to remove the vial transfer station 1118. In various embodiments, the vial transfer station 1118 includes a spike 1200, a station ID code 1120, a trigger 406, claws 408, 410, 412, 414, and a needle 416. FIG. 4A shows an embodiment of the vial transfer station 118 without a fluid pump device 102 attached on the underside. Some embodiments may also include a filter (not shown), similar to the filter 402 shown above. However, FIGS. 12A-12C are embodiments showing the fluid pump device 1202 attached to the fluid transfer station 1118.

The vial transfer station 1118 includes claws 408, 410, 412, 414 which receive the vial 122 and compression hold the vial 122 in place on the vial transfer station 118. In some embodiments, there are four claws 1208, 1210, 1212, 1214, as shown in the exemplary embodiment, however, in other embodiments, there may be more or less than four claws. In various embodiments, the claws 1208, 1210, 1212, 1214 spring back to receive the vial 122, and then when the vial 122 is removed, they spring back again to allow the removal of the vial 122 from the vial transfer station 1118.

Once the patient/caregiver confirms that the vial 122 is attached to the vial transfer station 1118, the UI 116 prompts the patient/caregiver to connect the UI 116 to the fluid pump device 1202. The patient/caregiver then connects the UI 116 to the fluid pump device 1202 by scanning the station ID code 120 in step 524. Next, in step 526, the UI 116 confirms there is a network connection between the fluid pump device 1202 and the UI 116. In some embodiments, the system confirms there is a BLUETOOTH® connection with the fluid pump device 1202 and the UI 116.

The methods described and shown above with respect to FIGS. 3-6A may also be used and applied to the embodiments of the single use system 1100 shown in FIGS. 11-13A.

In practice, the patient/caregiver removes the container cover 1104 when they are prompted to do so by the UI 116, which will occur once the patient/caregiver begins the therapy session and are ready to use the fluid pump device 1202. The container cover 1104 ensures that the container 1102 and all of its contents, i.e., the vial transfer station 1118, and the fluid pump device 1202, remain sterile until and unless use is desired. Thus, in various embodiments, the UI 116 may prompt the patient/caregiver to remove the container cover 1104 once confirmation of the therapy session and volume to be transferred from the drug for delivery 122 to the fluid pump device 1202 is confirmed by the patient/caregiver.

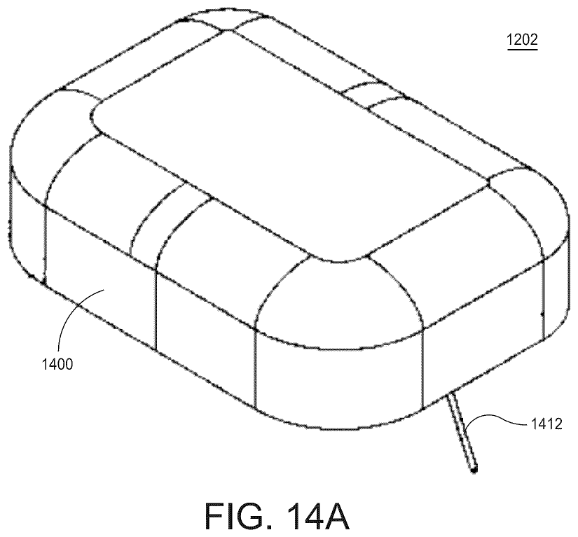

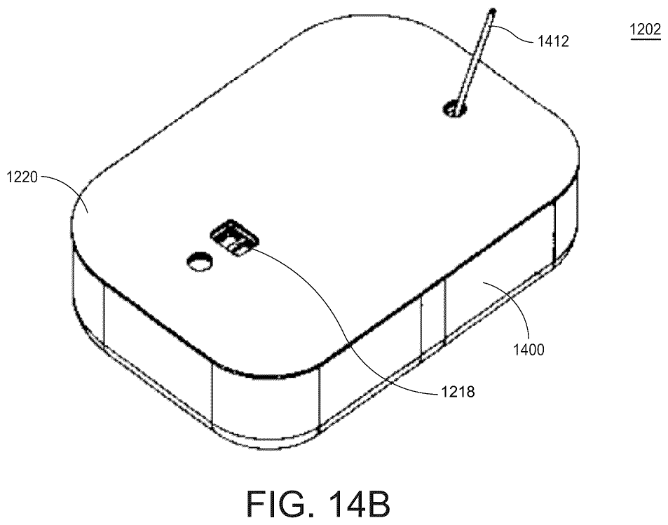

Referring now also to FIGS. 14A-23B, another exemplary embodiment of the fluid pump device 1202 is shown. In various embodiments, the fluid pump device 1202 is a disposable fluid pump device, and has exemplary dimensions of about 49.6×37.56×13.6 mm. However, in various other embodiments, the dimensions may vary. In embodiments where an adhesive 1220 is included, the dimensions may be about 50×38×14.14 mm. In some embodiments of the vial transfer station and fluid pump system, the dimension may be about 60×47×31.2 mm.

In various embodiments of the fluid pump device 1202 the pump outside casing or pump housing 1402 may be pliable and/or soft such that it conforms to a human body. In other embodiments, part of the pump housing 1402 may be pliable and/or soft while the remaining areas of the pump housing 1402 may be hard/non or less-pliable. In still other embodiments, the fluid pump device 1202 pump housing 1402 may be hard. In various embodiments, the size of the fluid pump device 1202 may be larger or smaller depending, e.g., on the intended use. In various embodiments, the intended use may require a larger reservoir. In these cases, the dimensions of the fluid pump device 1202 preferably may be expanded horizontally, while the vertical profile remains consistent. In some embodiments, the fluid pump device 1202 horizontal dimensions may be smaller. A smaller reservoir volume requirement allows for a smaller fluid pump device 1202 length. In some embodiments where the reservoir requirements are larger, the number and/or size of the batteries may be larger, allowing ample power to deliver the larger volume of drug in the reservoir. In some embodiments, the vertical dimensions may be larger than stated herein.

Still referring also to FIGS. 14A-22, in various embodiments, the fluid pump device 1202 pump housing 1402 includes a reservoir housing 1404 and a pump base housing 706. In some embodiments, the reservoir housing 1404 may be a soft, pliable material while the pump housing 1404 may also be in a soft, pliable material. However, in various embodiments, the reservoir housing 1404 and/or pump housing 1404 may be made from the same or different materials one from another, and/or either or both may be made from soft/pliable material and/or harder material.

In various embodiments, the reservoir 106 is attached to the underside of the reservoir housing 1404 (see 704, FIG. 8K), together with the reservoir port 1432, make up one embodiment of the pump cover (see FIG. 8K, 808). In some embodiments, the reservoir 106 is a plastic bag of a predetermined size. The size of the reservoir 106 may vary depending on many factors, including, but not limited to: the drug type and/or the therapy type and/or volume of drug to be delivered in the therapy session. In various embodiments, the reservoir 106 may hold 5 ml of fluid. However, in other embodiments, the reservoir 106 may hold between 2 ml-20 ml of fluid. In various embodiments, the reservoir 106 is composed of a plastic membrane that forms a sack/bag. The reservoir 106 includes a reservoir port 600, providing access to the needle 1216 for filling and to the fluid connection 114. However, in some embodiments, the reservoir may be pre-filled, e.g., by a pharmacist and/or by a pharmaceutical/drug company, and the reservoir 106 provided, already filled to the predetermined volume, to the patient/caregiver. In various embodiments, the reservoir 106 may be formed partially with the reservoir housing 1404, that is, the underside of the reservoir housing 1404 may serve as one side of the reservoir 106.

The reservoir port 1432 is also used to access the drug that was loaded into the reservoir 106 for delivery to the patient via a fluid connection 114. In some embodiments, which includes the exemplary embodiments shown and described herein, the drug is delivered from the reservoir 106 to the patient using a cannula 1412 connected to the reservoir 106 via the fluid connection 114. In various embodiments, the fluid path 108 within the fluid pump device 102 includes the path the fluid follows between the reservoir 106 and the cannula 1412.

In various embodiments, the reservoir 106 may be constructed from any material desired. In various embodiments the reservoir 106 may be constructed from material that is compatible with the drug/therapeutic in which is intended to be contained within the reservoir 106. In some embodiments, the reservoir 106 is constructed of EVA.

In various embodiments, the fluid pump device 1202 includes electronics 110 to pump the fluid from the drug for delivery/vial 122 to the reservoir 106 and from the reservoir 106 to the patient. In some embodiments, the electronics 110 include, but are not limited to, a PCB 1410 and batteries 1414, 1416 to control and power the motor 104. In various embodiments, the PCB 1410 may be an MCU with BLUETOOTH® function. However, in other embodiments, a different PCB 1410 may be used. In the exemplary embodiments, as shown and described herein, the batteries 1414, 1416 may be LR46 batteries or any other type of battery in the art. In various embodiments, the battery size and number may be selected based on many factors, including, but not limited to: amount of fluid to pump (both in the fill stage and delivery stage); the viscosity of the fluid; and the amount of time over which the therapy session/drug delivery is predetermined to occur. In the exemplary embodiment, the batteries 1414, 1416 include enough power to pump 5 ml of drug/therapeutic to a patient and to run the fluid pump device 102 for 3 days. However, in other embodiments, different batteries and different numbers of batteries may be selected provide the necessary power to deliver the intended therapy.

In various embodiments of the fluid pump device 1202 the fluid pump device 1202 may be reusable. In some of these embodiments, the batteries 1414, 1416 (or, in various embodiments, as discussed above, other sizes and/or a different number of battery/batteries may be used, but for the purposes of this disclosure, batteries 1414, 1416 refer to any one or more of battery/batteries in the fluid pump device 1202) may be rechargeable batteries 1414, 1416. In these embodiments, other elements may be included into the fluid delivery system 100 which may include, but are not limited to: a charging device; a charging station; and/or a charging port. All of these additional elements that may be added to the fluid delivery system 100 may collectively be referred to as peripheral charging element(s) 130 (see FIG. 1). In various embodiments, also included in the peripheral charging element(s) 130 may be a power cord and/or peripheral/external battery pack (not shown) that may power one of more devices shown and/or described with reference to one or more embodiments herein, e.g., a nebulizer and/or an IV pumping system (not shown).

In some embodiments, the fluid pump device 1202 may include an internal cannula 1412. As discussed above, in some embodiments, the cannula 1412 is automatically inserted into the patient once the fluid pump device 102 is adhered to/attached to the patient's skin. However, in various embodiments of the fluid pump device 102 an external cannula 1412 or other external fluid conduit to the patient, may be used. These include, but are not limited to, a cannula 1412 in fluid communication with the fluid pump device 102 via a predetermined length of tubing 206; a port connection to an IV line 206; and/or via a nebulizer (not shown) and a mask 204.

However, in various embodiments, a cannula 1412 may be housed within the pump base 1406 and include an assembly for cannula insertion that, in some embodiments, including the exemplary embodiments shown and described herein, may include an inserter cam 1438 powered by a spring 1418, and an inserter needle 1420 which wraps around the inserter cam 1438 and into a slider/carrier adapter 1446. In various embodiments, and as shown in FIG. 5C, in step 552, when the UI 116 receives confirmation from the patient/caregiver to “start infusion” in step 550, the fluid pump device 102 automatically inserts the cannula 112, 1412 and infusion beings. In various embodiments of this embodiment of the fluid pump device, method and system, it is in step 552 that the UI 116 sends a command to the fluid pump device 1402 PCB 1410 to trigger the inserter. Before this point, the spring 1418 is held in tension. When the trigger is given, the spring 1418 is released and the inserter is triggered to propel the inserter needle 1420 through the slider/carrier 1446 and into the cannula 1412, and both the inserter needle 1420 and cannula 1412 are inserted into the patient. At the end of this insertion motion, the inserter spring 1418 retracts, pulling the inserter needle 1420 out of the cannula 1412 and the patient, leaving the cannula 1412 inside the patient for subcutaneous delivery.

In various embodiments, the inserter needle 1420 is made from medical-grade stainless steel. In some embodiments, the cannula 1412 is made from medical-grade plastic. In some alternate embodiments, a stainless-steel cannula 1412 may be used, replacing the plastic cannula, and in these embodiments, a separate inserter needle 1420 is not needed. Thus, in these embodiments, and in some embodiments of these embodiments, using a slightly modified inserter system as the one shown and described herein, the stainless-steel cannula may be wrapped around the inserter cam 1438 and the spring 1418 propels the cannula 1412 into the patient, but does not retract. Thus, the steel cannula remains in the patient and delivery/infusion is completed using the stainless-steel cannula. In various embodiments, the stainless-steel cannula system described herein may be preferably used only where the drug/therapeutic being delivered is of a low/lower viscosity. However, in other embodiments, a stainless-steel cannula may be used for the delivery/infusion or any fluid/drug/therapeutic.

Referring now to FIGS. 1-22, in various embodiments of the fluid pump device 102 a motor 105 is used to pump fluid from the drug for delivery 122 (e.g. vial 122) to the reservoir 106, and from the reservoir 106 to the patient via the fluid conduit to the patient 112. An exemplary embodiment of the motor 104 used in the fluid pump device 104 includes a peristaltic pump 1434. In other embodiments, the motor 104 may be any device or combination of devices that work to move fluid from the drug for delivery 122 to the reservoir 106 and from the reservoir 106 to the patient. Other embodiments of motors 104 which may be used for the fluid pump device 102 include, but are not limited to: another embodiment of a peristaltic pump; a membrane pump; a syringe pump; and/or any positive displacement pump, and/or any motor known in the art.

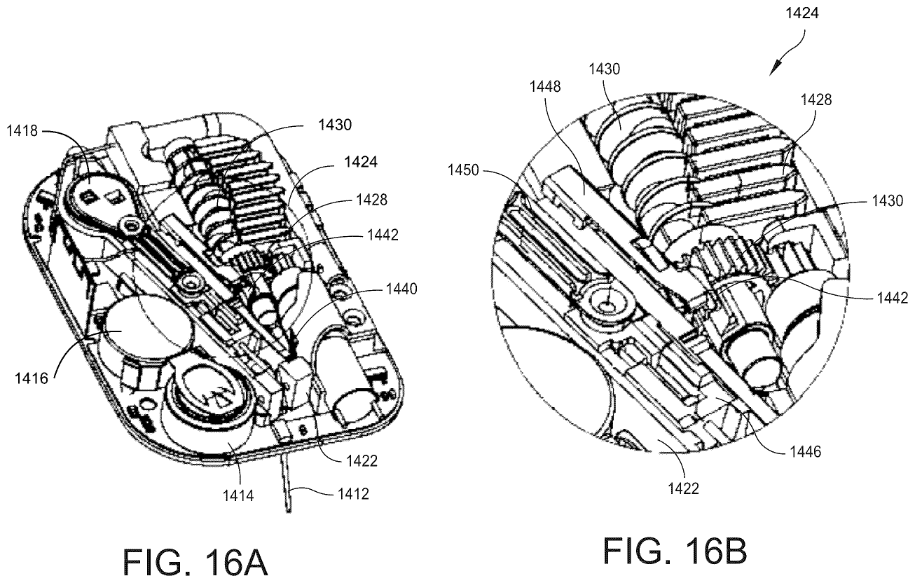

Referring now also to FIGS. 16A-20, the peristaltic pump 1434 disclosed herein is an exemplary embodiment of the motor 104. In the exemplary embodiments, the peristaltic pump 1434 includes a driver motor 1424 that causes the crankshaft 1430 to rotate. The crankshaft 1430 interacts with the pump fingers 1428 to cause the pump fingers 1428 to interact and effect the pump tubing 1426. The pump fingers 1428 cause the fluid to be pumped from the reservoir 106 to the cannula 112, 1412 at a prescribed flow rate. The flow rate may be varied, e.g., by varying the rotational speed of the crankshaft 1430.

In various embodiments, the drive motor 1424 is a DC motor, and any DC motor known in the art may be used. The crankshaft 1430 is designed such that it interacts with the pump fingers 1428 such that the plurality of the pump fingers 1428 are articulated at different moments, during the full rotation of the crankshaft 1430, to exert pressure onto the pump tubing 1426 and move the drug/fluid/therapeutic from the reservoir 106 to the fluid conduit to the patient 112, 1412.

In various embodiments, the pump tubing 1426 is made from PVC, however, in other embodiments, the pump tubing 1426 may be made from any material desired. In the exemplary embodiment, the PVC pump tubing 1426 provides a high resistance and therefore, a more accurate volume of fluid/drug/therapeutic may be pumped from the reservoir 106 to the fluid conduit to patient 112, 1412 per rotation of the crankshaft 1430. Additionally, the PVC pump tubing 1426 resistance allows for higher viscosity fluids/drugs/therapeutics to be pumped using the fluid pump device 102. This may be beneficial/desirable for many reasons, including, but not limited to, the ability to deliver monoclonal antibody treatments and other drug therapies that include high viscosity fluids/drugs/therapeutics. As the PVC pump tubing 1426 is more rigid than, e.g., a membrane or other, less rigid material, the pump tubing 1426 will maintain its integrity throughout the fluid pump device 102 life/while in use. The PVC pump tubing 1426 together with the peristaltic pump 1434 allows for high torque pumping and high volume pumping, i.e., pumping fluid/drug/therapeutic at 20 psi and at a rate of 200 ml/hour, or 5 cc in less than 2 minutes.

Still referring to FIGS. 14A-22, in various embodiments, the peristaltic pump 1434 shown and described herein has many benefits in addition to those discussed above. In the exemplary embodiment, the drive motor 1424 is powerful enough to pump fluid/drug/therapeutic as described above, however, is small enough that it does not require a high level of power to turn the crankshaft 1430. Thus, a higher volume of fluid/drug/therapeutic may be pumped by the peristaltic pump 1434 (and the fluid pump device 1202) using a low amount of power. This may be beneficial/desirable for many reasons, including, but not limited to: ability to pump a higher volume of fluid/drug/therapeutic using smaller batteries/less power; allowing for longer therapies to be delivered by the fluid pump device 1202 using a single charge of the batteries; requiring smaller batteries therefore allowing for a smaller footprint design for the pump housing 1402 and overall footprint and size/dimensions of the fluid pump device 1202. A smaller profile is beneficial/desirable for many reasons, including, but not limited to: providing a lighter/smaller pump may be more easily worn by a patient; providing a lighter/smaller pump may be more easily attached to an IV line 206 or nebulizer and mask 204 such that a patient may be ambulatory while receiving therapies delivered by the fluid pump device 1202, including being ambulatory while receiving fluid/drug/therapy from the fluid pump device 1202 while also connected to an IV line 206.

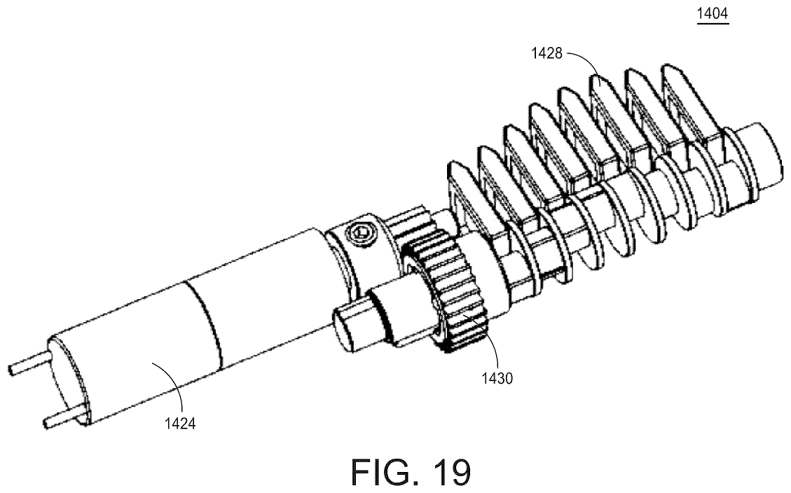

Referring now also to FIGS. 19-20, the peristaltic pump 1434 shown and described herein is an exemplary embodiment of the peristaltic pump 1434 for the fluid pump device 1202. This design of the exemplary peristaltic pump 1434 is more power efficient compared with a prior art peristaltic pump. As may be seen from inspection of FIG. 23, the power consumption 2302 of the peristaltic pump 1434 described and shown herein as the exemplary peristaltic pump 1434 is substantially lower compared with the power consumption 2300 of the prior art peristaltic pump 2300.

Still referring also to FIGS. 14A-23, in the exemplary embodiments, the peristaltic pump 1434 may be more durable and reliable for many reasons, including, but not limited to, that the peristaltic pump 1434 does not include a valve. The peristaltic pump 1434, in operation, always includes where one pump finger 1428 is placing force on the pump tubing 1426. This acts in a similar manner as a valve, however, has many advantages, including, but not limited to: performing as a safe, accurate, and reliable pump with less parts, thus may be constructed lighter and smaller than other pumps that perform similar functions, and still pump at 20 psi. In various embodiments of the peristaltic pump 1434 the pump fingers 1428 include a design where the point of contact on the pump tubing 1426 is triangularly shaped. This provides for more pressure exertion on the pump tubing 1426 as compared with a flat or rounded surface, however, does not require addition power to exert a higher force. Thus, the exemplary embodiment of the peristaltic pump 1434 shown and described herein includes the ability to pump fluid/drug/therapeutic at a higher psi, e.g., 20 psi, which allows for the pumping of higher viscosity drug/fluid/therapeutic to be delivered, and at higher volumes for unit time.

The peristaltic pump 1434 shown herein is an exemplary embodiment for a patch-sized fluid pump device 1202. However, the design of the peristaltic pump 1434 may be scaled larger or smaller depending on the type of drug/fluid/therapeutic and/or therapy being delivered using the fluid pump device 1202. Thus, e.g., if larger volumes of drug/fluid/therapeutic are desired to be pumped/delivered to a patient in a short amount of time, a faster flow rate may be desired. The size of the peristaltic pump 1434, including the size of the pump fingers 828, crankshaft 830, and drive motor 1424 may be increased to reach a desired pumping volume/aliquot per rotation of the crankshaft 1430. Conversely, the peristaltic pump 1434 may be scaled down to accommodate very small delivery volumes and/or delivery of a single aliquot over, e.g., a short period of time, which may include, but is not limited to, less than 1 minute to 5 minutes. In the exemplary embodiments, the peristaltic pump 1434 pumps 0.02 ml-ml per rotation.

In various embodiments of the peristaltic pump 1434, there is a sensor (not shown) that counts the crankshaft 1430 rotations. That same sensor provides a feedback loop to a CPU located on the PCB 1410 which, in various embodiments, validates that the crankshaft 1430 actually rotated, i.e., that the crankshaft 1430 is functioning, after current is applied to the drive motor 1424. Thus, both while transferring fluid from the vial 122 to the reservoir 106, and while delivering/infusing the fluid from the reservoir 106 to the patient via a fluid conduit to the patient 112, the UI 116 receives confirmation from the sensor (via the CPU) of the number of rotations of the crankshaft 1430 that are completed. Based on this sensor data, the system, in various embodiments, may calculate the volume pumped. In various embodiments, this sensor and feedback loop allows the UI 116 to determine when the fluid transfer is complete (step 530, FIG. 5A) and when infusion is complete (steps 556, 558, FIG. 5C). Thus, the UI 116 may verify that the volume requested by the patient/by a preprogrammed therapy for infusion to the patient has been delivered, and that the volume of fluid requested for transfer from the drug for delivery 122 (e.g., vial 122) has been transferred to the reservoir 106.

In various embodiments, the peristaltic pump 1434 may be beneficial/desirable for additional reasons, including, but not limited to: if air is in the pump tubing 1426, the peristaltic pump 1434 will still be able to pump the drug/fluid/therapeutic. Thus, air bubbles are mitigated as a consequence of the design of the motor 104 used in the exemplary embodiments of the fluid pump device 1202.

In various embodiments, the fluid pump device 1202 includes an occlusion sensor. In some embodiments, the occlusion sensor may be a pressure sensor or an optic sensor. The location of the occlusion sensor may be anywhere along the fluid path, including, but not limited to, where the drug/fluid/therapeutic enters the cannula 1412 or other fluid conduit to the patient.

In some embodiments of embodiments including a nebulizer, an ETO2 sensor may be embedded into the fluid pump device 1202. This may be beneficial/desirable for many reasons, including, but not limited to measuring the outcome of the nebulized medication delivery.

In various embodiments of the fluid delivery system 100 shown and disclosed herein, communications between the fluid pump device 102 and the UI 116 are ongoing for many reasons, including, but not limited to: for ensuring patient safety and compliance with prescribed therapy.

In various embodiments, a patient portal may be setup through the source of the drug/fluid/therapeutic, e.g., a drug company/pharmaceutical company. The UI 116 is a patient “app” that may be on any personal electronic device, e.g., a smartphone. As discussed in more detail above, the patient/caregiver uses the UI 116 to scan both the drug 122 and the vial transfer station and fluid pump system 422. This information may be shared with the patient portal and a confirmation may be made that the patient/caregiver is using not only the correct drug 122 and fluid pump device 102, but that neither is a counterfeit. Thus, scanning these elements of the system serves to ensure patient safety and that the correct therapy will be delivered to the correct patient. In various embodiments, the fluid delivery system 100 will not allow the filling steps until and unless both the drug 122 and the vial transfer station and fluid pump system 422 are authenticated.

In various embodiments, once the vial transfer station and fluid pump system is scanned, this information is sent to a server to indicate that the fluid pump device 1202 in that vial transfer station and fluid pump system has been used. This is beneficial/desirable for many reasons, including to prevent re-use which may be dangerous to a patient; and/or to confirm compliance by the patient that the prescribed therapy has been completed.

In various embodiments, communications between the fluid pump device 102, 1202 and the UI 116 are completed via a cloud-based device gateway. Fleet management service software communicates to the communication module of the UI 116 to connect the fluid pump device. The connection to the fluid pump device 102, 1202 is separate and maintained by the conduit app/UI 116. Thus, communication to the fluid pump device 102, 1202 and the service side, e.g., authentication, of the system 100 may be accomplished.

Referring now also to FIG. 15G, in various embodiments of the fluid pump device 1202, the fluid pump device 1202 may include a reflective object sensor 1436. The reflective object sensor 1436 determines/senses with which type of surface the fluid pump device 1202 is in contact. In various embodiments, the reflective object sensor 1436 determines whether the fluid pump device 1202 is in contact with, e.g., patient skin or the vial transfer station 1118. If the reflective object sensor 1436 senses that the fluid pump device 1202 is in contact with patient skin, then the fluid pump device 1202 insertion/cannulation system may be triggered. If the reflective object sensor 1436 senses that the fluid pump device 1202 is in contact with the vial transfer station 1118, then the insertion/cannulation system may not be triggered. This may be beneficial/desirable for many reasons, including, but not limited to, preventing “misfires”, i.e., accidental/non-intentional triggering of the insertion/cannulation system. Thus, in various embodiments, the insertion/cannulation system may not be triggered until and unless the reflective object sensor 1436 detects that the fluid pump device 1202 is in contact with human skin/patient skin. Thus, while the fluid pump device 1202 is in contact with the vial transfer station 1118, the insertion/cannulation system may not be triggered. In various embodiments, any reflective object sensor may be used, including, but not limited to, the reflective object sensor from TT Electronics, UK, part numbers: OPB606A, OPB606B, OPB606C, OPB607A, and/or OPB607C.

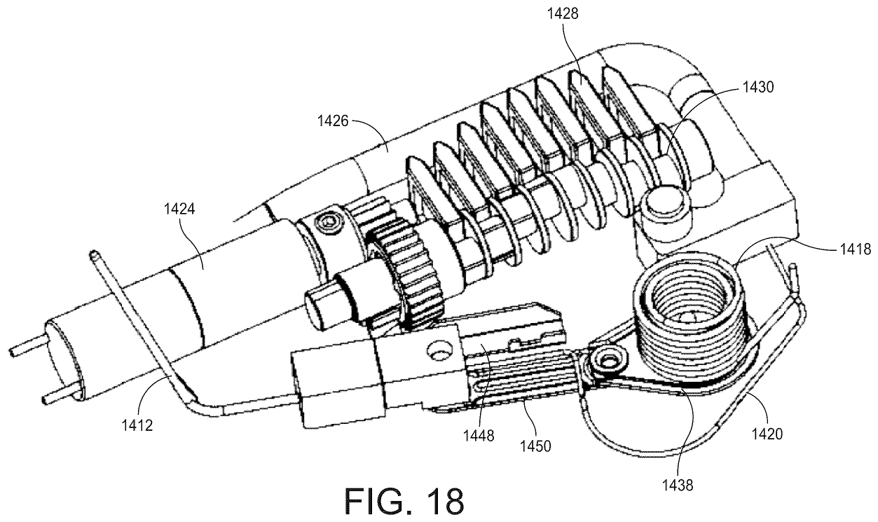

Referring now also to FIGS. 18-20, an embodiment of the drive motor 1424 and peristaltic pump crankshaft 1430 and pump tubing 1426 is shown. This design of the exemplary peristaltic pump 104 is more power efficient compared with a prior art peristaltic pump. As may be seen from inspection of FIG. 9, the power consumption 902 of the peristaltic pump 104 described and shown herein as the exemplary peristaltic pump 104 is substantially lower compared with the power consumption 900 of the prior art peristaltic pump 900.

Cannulation Systems

Referring now also to FIGS. 15B-17, one embodiment of the inserter system is shown. The inserter system includes an inserter cam 1438, an inserter spring 1418, a needle guide 1422, a slider/carrier 1446, a beveled protrusion 1440, a stopper 1442, a latch 1444, a release lever 1448, and link arm 1450.

The drive motor 1424 rotates the crankshaft 1430 counter clockwise during the filling process. The cannula 1412 is prevented from being inserted until the drive motor 1424 is commanded to rotation clockwise. The inserter therefore is prevented from being triggered, and therefore the cannula 1412 is prevented from being inserted, until a command is made to begin infusion. Referring again also to FIG. 5C, in some embodiments, this is done in step 550 when the patient/caregiver indicates “start infusion” on the UI 116. As discussed herein, in some embodiments, insertion may begin when the reflective object sensor detect (or sends a signal that indicates) that the fluid pump device 1202 is located against skin. In some embodiments, the insertion will begin once this signal is received. In other embodiments, the insertion or infusion will not begin until and unless a signal is received (by the UI or give directly) that the fluid pump device 1202 is located on skin. This may be beneficial/desirable for many reasons, including, but not limited to, that non-intentional insertion may be prevented until and unless the fluid pump device 1202 is located on an appropriate/desired surface, e.g., skin (or the patient's skin).

Once the command is given, the drive motor 1424 rotates counterclockwise. This rotation releases the link arm 1450 which is locked in place/unable to otherwise spring upwards by the beveled protrusion 1440 on the drive motor 1424. The release of the link arm 1450 from the beveled protrusion 1440 on the drive motor 1424 also removes the link arm 1450 from contact with the latch 1444 on the crankshaft 1430 and allows the crankshaft to rotate in the clockwise direction, thus allowing infusion.

The release of the latch arm 1450 also triggers the releases of the inserter cam 1438, rotation of the link arm 1450 and release of the slider/carrier 1446. The torsion inserter spring 1418 provides the force to propel the slider/carrier 1446 which carries the inserter needle 1420 in a lateral motion that propels it along the needle guide 1422 towards the patient, allowing the insertion of the cannula 1412 into the patient.

In this embodiment, the insertion needle 1420 is located within the cannula 1412, thus, when the insertion needle 1420 is propelled/forced along the needle guide 1422 and into the patient, this introduces the cannula 1412 into the patient's subcutaneous skin region.

The inserter spring 1418 and also the inserter cam 1438 rotates in the opposite direction/snaps back. This causes the inserter needle 1420 (sometimes referred to as an insertion needed) to be retracted from the cannula 1412 and the patient.

Thus, after the insertion of the cannula 1412, the inserter needle 1420 is retracted. In various embodiments, the slider/carrier 1446 is also retracted. In some embodiments, the slider/carrier 1446 includes a needle septum (not shown) which seals after the inserter/insertion needle 1420 is retracted.

In various embodiments, the retraction of the slider/carrier 1446 allows the fluid path (not shown) to be open between the reservoir 106, thus allowing the flow and infusion of the drug for delivery 122 between the reservoir 106 and the patient 200 and or between the reservoir 106 and the cannula 1412.

The inserter system may be beneficial/desirable for many reasons, including, but not limited to, that the drive motor 1424 is prevented from turning clockwise by the release lever 1448 position against the beveled protrusion 1440 located on the drive motor 1424. Thus, until and unless the UI 116 commands the drive motor 1424 to rotate clockwise, releasing the release lever 1448 from its position against the beveled protrusion 1440 on the drive motor 1424, the crankshaft 1430 may not rotate clockwise, and therefore, the fluid pump device 1202 cannot deliver drug for delivery 122 to the patient 200. Thus, this is a safety mechanism to prevent unintentional delivery to the patient 200 by requiring a confirmation from the user/caregiver to the UI 116 that insertion is desired, and therefore, that infusion/drug delivery is desired. Additionally, this design prevents unintentional triggering or release of the insertion/inserter needle 1420 during filling and not until the user/caregiver is ready to begin infusion/insert the cannula 1412.

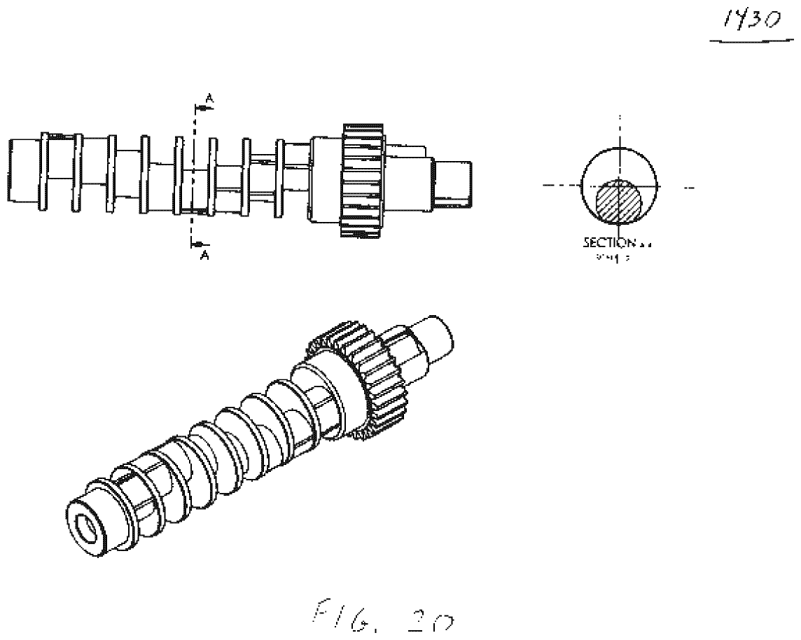

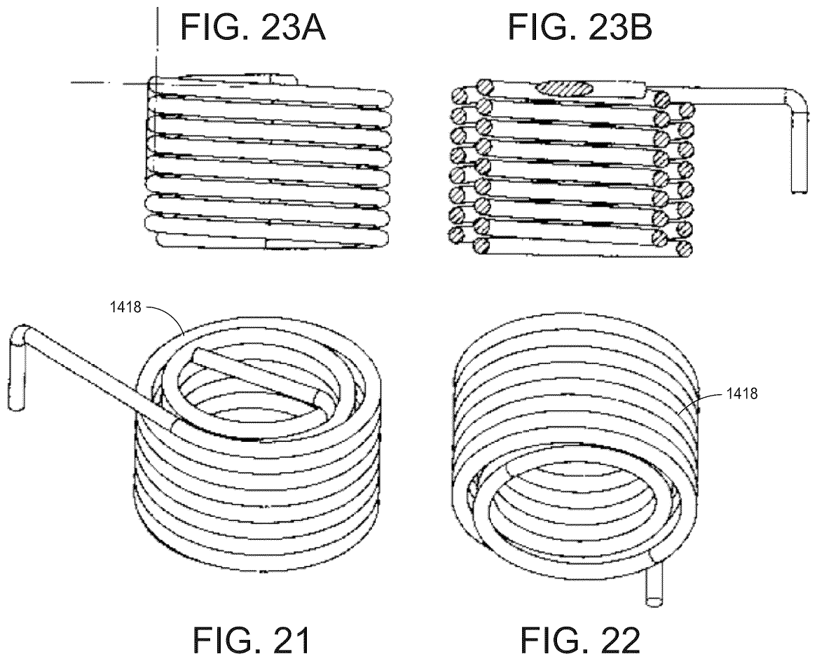

Referring now also to FIGS. 21-23B, various views of the inserter spring 1418 (or insertion spring 1418) are shown. In various embodiments, the inserter spring 1418 is a double layer inside and outside design. This may be beneficial/desirable for many reasons, including, but not limited to, the design increases the working torque of the tension inserter spring 1418, and increases the working angle. In various embodiments, any torsion spring may be used within any of the inserter designs shown and/or described herein.

Although multiple fluid pump devices and inserter systems are shown and disclosed herein, it should be understood that elements from any embodiment of a fluid pump device may be used together with any inserter system, and therefore, the embodiments as shown are exemplary, and other embodiments are included in the breadth and scope of this disclosure. This includes using any embodiments of a fluid pump device with any embodiment of an inserter system.

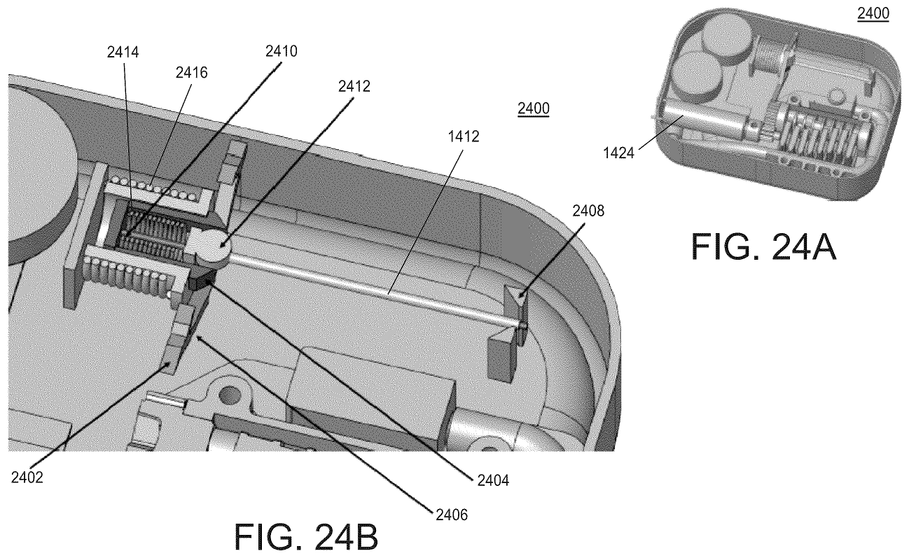

Referring now also to FIGS. 24A-24B, another embodiment of an inserter for any one or more of the fluid pump device disclosed herein is shown. This embodiment of an inserter 2400 includes an actuation motor link 2402, an actuation retainer 2406, a needle catch 2404, a catch release 2408, an inserter/insertion needle 2410, a cannula fluid connection 2412, a first spring 2414, and a second spring 2416. FIG. 24A shows the inserter system 2400 in the pre-deployed position and FIG. 24B shows the inserter system 2400 in the post-deployed position.

In this embodiment, in the pre-deployment position, after the drive motor 1424 moves in the counterclockwise position to fill the reservoir 106, the drive motor 1424 reverses, to the counterclockwise position, to begin the insertion process (and following, the infusion process). This counterclockwise movement of the drive motor 1424 activates the activation motor linkage 2402 and deforms the living hinge actuation retainer 2406. The insertion/inserter needle 2410 and cannula 1412 translate into the patient's skin, powered by the first spring 2414 allowing the needle catch 2404 to disengage. The needle return spring/second spring 2416 retracts the insertion/inserter needle 2410.

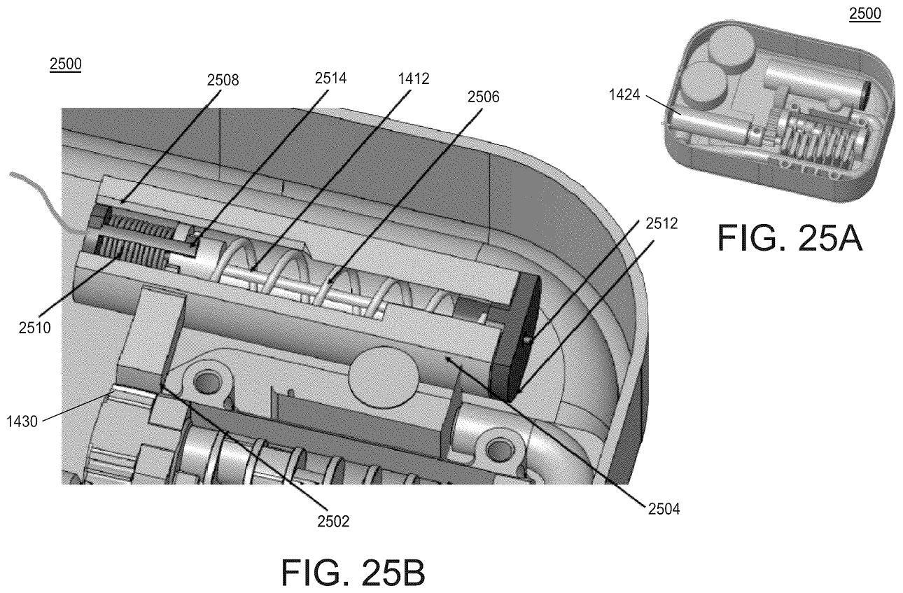

Referring now also to FIGS. 25A-25B, another embodiment of an inserter system is shown. In various embodiment, the sleeve inserter 2500 includes a partial gear segment 2502, a retention and actuation sleeve 2504, an extension spring 2506, a cannula 1412, an actuation cam slot 2508, a needle return spring 2510, a crankshaft 1430, an inserter/insertion needle 2512, and a needle retainer 2514. In various embodiments, during pre-deployment (FIG. 25A), the drive motor 1424 revolves in the counterclockwise direction to fill the reservoir 106. The retention and actuation sleeve 2504 is stationary during fill. The drive motor 1424 then reverses for insertion and the retention and actuation sleeve 2504 engages the partial gear segment 2502 to release the insertion/inserter needle cannula catch/needle retainer 2514. Using the force from the torsion extension spring 2506, the needle 2512 and canula 1412 are translated into the patient's body, and the cam slot disengage the needle retainer 2514. The needle return spring 2510 retracts the needle 2512.

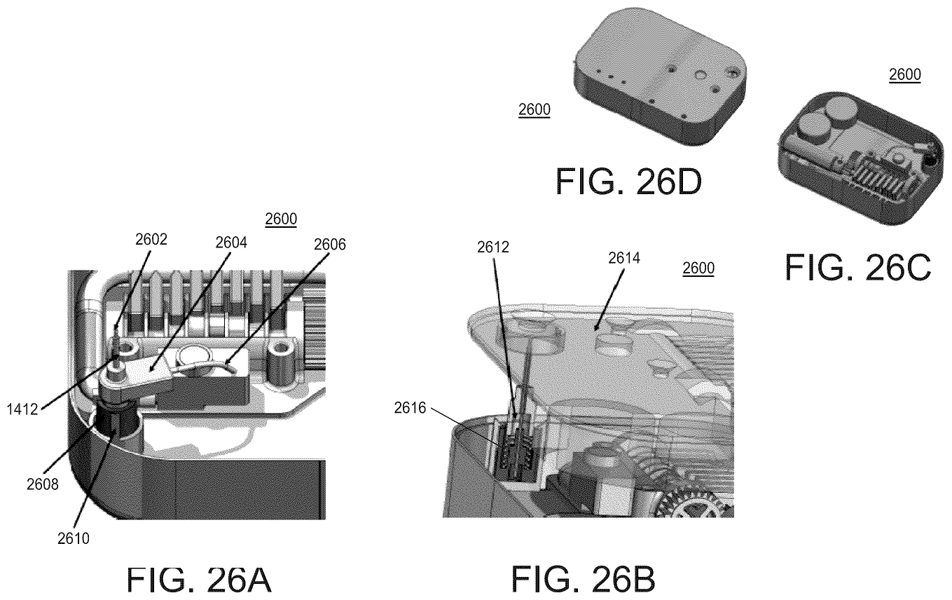

Referring now also to FIGS. 26A-26B, a 90-degree inserter system 2600 is shown. The system 2600 includes an inserter needle 2602, cannula 1412, needle holder 2608, needle catch 2610, needle port 2604, fluid path 2606, sealing septum 2612, and a housing 2614. In this system 2600, pre-deployment (FIG. 26A), a user/caregiver pushes/exerts manual force upon the device 2600 onto the user's skin to insert the insertion/inserter needle 2602 and the cannula 1412. Post deployment of the insertion/inserter needle 2602, the needle catch 2610 opens to allow a compression spring 2616 to retract the insertion/inserter needle 2602. The insertion/inserter needle 2602 then comes out of the fluid path 2606. The cannula 1412 remains in the user's body/skin.

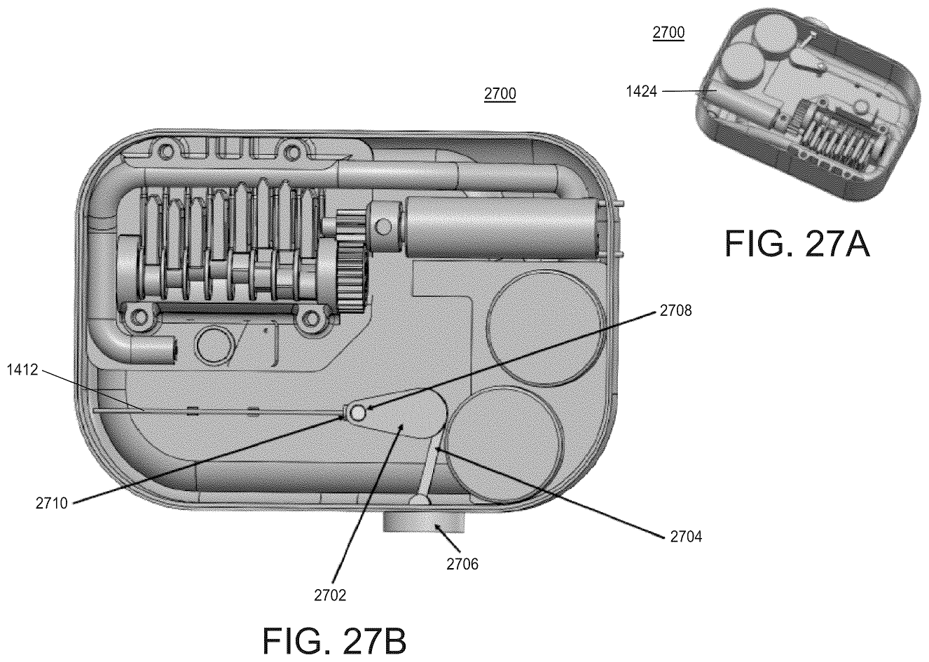

Referring now also to FIGS. 27A-27B, another embodiment of an inserter system 2700 is shown 2700. The cam wheel deployment inserter system 2700 includes a cam wheel 2702, a locking mechanism 2704, a release button 2706, a torsion spring 2708 and a needle and cam follower 2710. In pre-deployment (FIG. 27A), the cam wheel 2702 is in the retracted position, and the locking mechanism 2704 is engaged. During deployment (FIG. 27B), the release button 2706 is pressed (by a user/caregiver), disengaging the locking mechanism 2704. In the deployment position, the needle (not shown, is located within the cannula 1412) is translated using the cam wheel 2702. The cam wheel 2702 continues travel for a full rotation, retracting the needle (not shown) in one motion.

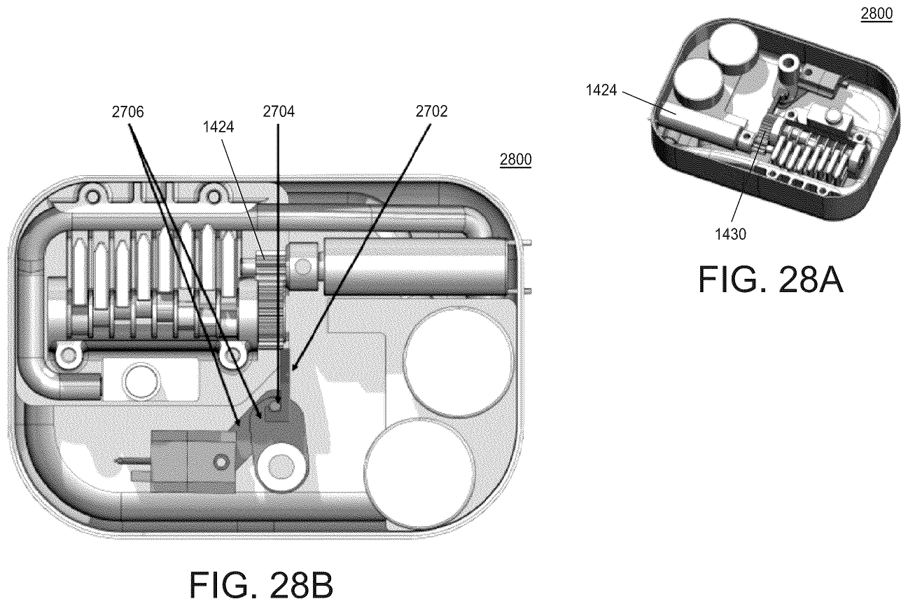

Referring now also to FIGS. 28A-28B, another embodiment of an inserter system 2800 is shown 2800. The rotating arms inserter system 2700 includes the drive motor 1424, retaining arm 2702, retaining pin 2704, and rotating arms 2706 (needle and cannula are not shown in detail in these FIGS., however, in various embodiments, they are similar to those disclosed, shown, and/or described elsewhere herein). During pre-deployment (FIG. 28A), the drive motor 1424 rotates clockwise and the retaining arm 2702 stays in place. Upon deployment (FIG. 28B), the drive motor 1424 rotates counterclockwise, and the retaining arm 2702 moves down to allow deployment of the insertion needle and cannula (not shown). The needle and cannula (not shown) are deployed. The rotating arms 2706 continue to rotate to retract the needle (not shown).

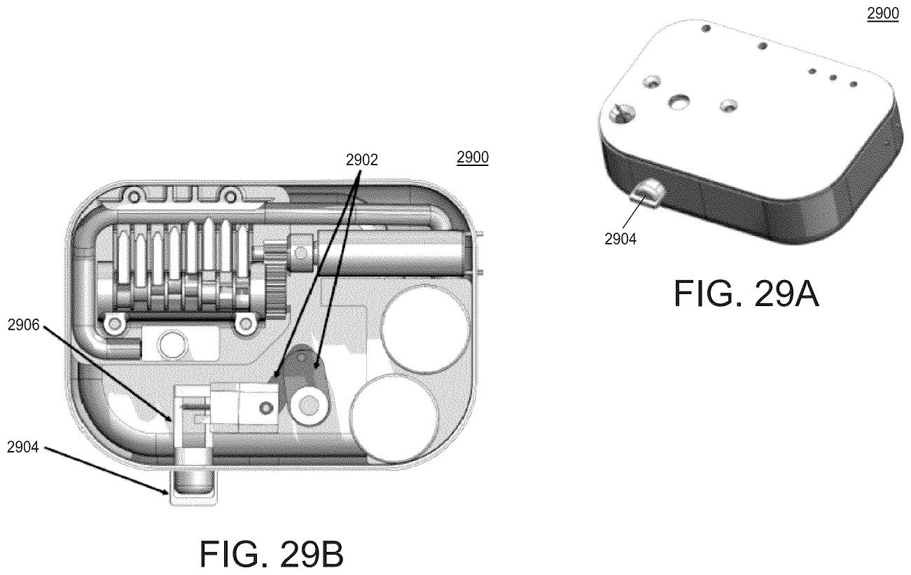

Referring now also to FIGS. 29A-29B, another embodiment of an inserter system 2900 is shown 2900. The rotating arms manual inserter system 2900 includes rotating arms 2902, a safety tab 2904, and release button 2906. During pre-deployment, a user/caregiver removes the safety tab 2904. During deployment, the user/caregiver pushes the release button 2906. The needle and cannula (not shown) are deployed. The rotating arms continue to rotate to retract the needle (not shown, however, the various embodiments of needles and/or cannulas shown and/or described herein may be used in this embodiment).

The computer readable medium as described herein can be a data storage device, or unit such as a magnetic disk, magneto-optical disk, an optical disk, or a flash drive. Further, it will be appreciated that the term “memory” herein is intended to include various types of suitable data storage media, whether permanent or temporary, such as transitory electronic memories, non-transitory computer-readable medium and/or computer-writable medium.

It will be appreciated from the above that the invention may be implemented as computer software, which may be supplied on a storage medium or via a transmission medium such as a local-area network or a wide-area network, such as the Internet. It is to be further understood that, because some of the constituent system components and method steps depicted in the accompanying Figures can be implemented in software, the actual connections between the systems components (or the process steps) may differ depending upon the manner in which the present invention is programmed. Given the teachings of the present invention provided herein, one of ordinary skill in the related art will be able to contemplate these and similar implementations or configurations of the present invention.

It is to be understood that the present invention can be implemented in various forms of hardware, software, firmware, special purpose processes, or a combination thereof. In one embodiment, the present invention can be implemented in software as an application program tangible embodied on a computer readable program storage device. The application program can be uploaded to, and executed by, a machine comprising any suitable architecture.

While various embodiments of the present invention have been described in detail, it is apparent that various modifications and alterations of those embodiments will occur to and be readily apparent to those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having,” and variations thereof herein, is meant to encompass the items listed thereafter and equivalents thereof as well as additional items while only the terms “consisting of” and “consisting only of” are to be construed in a limitative sense.

The foregoing description of the embodiments of the present disclosure has been presented for the purposes of illustration and description. It is not intended to be exhaustive or to limit the present disclosure to the precise form disclosed. Many modifications and variations are possible in light of this disclosure. It is intended that the scope of the present disclosure be limited not by this detailed description, but rather by the claims appended hereto.

A number of implementations have been described. Nevertheless, it will be understood that various modifications may be made without departing from the scope of the disclosure. Although operations are depicted in the drawings in a particular order, this should not be understood as requiring that such operations be performed in the particular order shown or in sequential order, or that all illustrated operations be performed, to achieve desirable results.

While the principles of the disclosure have been described herein, it is to be understood by those skilled in the art that this description is made only by way of example and not as a limitation as to the scope of the disclosure. Other embodiments are contemplated within the scope of the present disclosure in addition to the exemplary embodiments shown and described herein. Modifications and substitutions by one of ordinary skill in the art are considered to be within the scope of the present disclosure.

What is claimed:

1. A fluid pumping system comprising:
a fluid pump device comprising:
a pump housing;
an adhesive located on the outside of the pump housing; and
an adhesive cover attached to the adhesive; and
a vial transfer station, wherein the adhesive cover is attached to the vial transfer station;
wherein when the fluid pump device is removed from the vial transfer station, the adhesive cover is removed from the adhesive and the adhesive cover remains attached to the vial transfer station.

2. The fluid pumping system of claim 1, wherein the fluid pump device further comprising a drive motor,
wherein when the drive motor rotates in a first direction, the drive motor actuates filling a reservoir with a drug for delivery, and
wherein when the drive motor rotates in a second direction, the drive motor actuates infusion the drug for delivery between the reservoir and a cannula.

3. The fluid pumping system of claim 2, further comprising:
an automatic cannula inserter comprising:
an inserter cam;
a torsion inserter spring;
a release lever; and
a link arm,
wherein the release lever prevents the drive motor from rotating in the second direction.

4. The fluid pumping system of claim 3, further comprising wherein when the automatic cannula inserter is triggered, the release lever allows the drive motor to rotate in the first direction.

5. The fluid pumping system of claim 3, wherein the fluid pump device comprising a reflective object sensor that sends signals to a user interface.

6. The fluid pumping system of claim 5, wherein when the reflective object sensor sends a signal to the user interface indicating the fluid pump device is in contact with human skin, the user interface sends a signal to trigger the automatic cannula inserter.

7. A fluid pump device comprising:
a filling state and an infusion state;
a drive motor;
a crankshaft connected to the drive motor, the drive motor for rotating the crankshaft;
a pump tubing; and
a peristaltic pump comprising:
a plurality of pump fingers,
wherein one of the plurality of pump fingers exerts force on the pump tubing at all times once the infusion state is initiated.

8. The fluid pump device of claim 7, wherein the plurality of pump fingers comprising a triangular shape located at a point of contact with the pump tubing.

9. The fluid pump device of claim 7, further comprising a user interface in communication with the fluid pump device.

10. The fluid pump device of claim 9, wherein the user interface comprising a pre-programmed pump rate of infusion.

11. The fluid pump device of claim 7, further comprising:
a pump cover comprising an outside and an underside, wherein a reservoir is attached to the underside of the pump cover.

12. The fluid pump device of claim 11, wherein the reservoir is pre-filled with a drug for delivery by the fluid pump device.

13. The fluid pump device of claim 7, further comprising a pump base comprising:
an automatic cannula inserter comprising:
an inserter cam;
a torsion inserter spring;
a release lever; and
a link arm,
wherein the release lever prevents the drive motor from rotating in the second direction.

14. A fluid pumping system comprising:
a fluid pump device comprising:
a pump housing;
an adhesive located on the outside of the pump housing;
an adhesive cover attached to the adhesive;
a reflective object sensor; and
an automatic inserter;
a vial transfer station, wherein the adhesive cover is attached to the vial transfer station;
wherein when the fluid pump device is removed from the vial transfer station, the adhesive cover is removed from the adhesive and the adhesive cover remains attached to the vial transfer station; and
a user interface in remote communication with the fluid pump device.

15. The fluid pumping system of claim 14, wherein when the user interface receives a signal from the reflective object sensor that the pump housing is attached to human skin, the automatic inserter is triggered.

16. The fluid pumping system of claim 14, wherein the fluid pump device comprising a filling state and an infusion state.

17. The fluid pumping system of claim 16, wherein the vial transfer station comprising a station ID, wherein the user interface receives the station ID prior to initiation of the fluid pump device filling state.

18. The fluid pumping system of claim 14, wherein the fluid pump device further comprising a drive motor,
wherein when the drive motor rotates in a first direction, the drive motor actuates filling a reservoir with a drug for delivery, and
wherein when the drive motor rotates in a second direction, the drive motor actuates infusion the drug for delivery between the reservoir and a cannula.

19. The fluid pumping system of claim 18, wherein the fluid pump device further comprising:
an automatic cannula inserter comprising:
an inserter cam;
a torsion inserter spring;
a release lever; and
a link arm,
wherein the release lever prevents the drive motor from rotating in the second direction.

20. The fluid pumping system of claim 19, further comprising wherein when the automatic cannula inserter is triggered, the release lever allows the drive motor to rotate in the first direction.
